(19) United States
(12) Patent Application Publication
(10) Pub. No.: US 2026/0369358 A1
(43) Pub. Date: Nov. 19, 2026

(54) GENERATING A GENE SIGNATURE FROM A SIGNATURE DISCOVERY PIPELINE THAT USES CUSTOM DIFFERENTIAL EXPRESSION ANALYSIS

(71) Applicant: Recursion Pharmaceuticals, Inc., Salt Lake City, UT (US)

(72) Inventors: Andrew Simon WALKER, Didcot (GB); Otto Kit MORRIS, Reading (GB)

(21) Appl. No.: 19/212,240

(22) Filed: May 19, 2025

(51) Int. Cl. G16B 35/00; C12N 15/10; G16B 40/20

(52) U.S. Cl. CPC G16B 35/00; C12N 15/1089; G16B 40/20

(57) ABSTRACT

The present disclosure relates to systems, non-transitory computer-readable media, and methods that generate a gene signature from a signature discovery pipeline that uses custom differential expression analysis for a target compound. Indeed, in one or more implementations, the disclosed systems generate a first set of gene differential expression measures and a second set of gene differential expression measures from applying a target compound to first pre-clinical dataset and a second pre-clinical dataset (corresponding to different data modalities). Moreover, the disclosed systems generate a multi-modal ranked set of genes from the gene differential expression measures. Furthermore, the disclosed systems generate a gene signature from the multi-modal ranked set of genes.

### BACKGROUND

Recent years have seen significant developments in hardware and software platforms for identifying and developing drug compounds to treat certain diseases. For example, conventional systems have developed complex methods for filtering through large sets of data and homing in on relevant biological targets (e.g., for a particular disease). In particular, conventional systems attempt to develop more personalized/tailored drug compounds to treat/target diseases. Despite these recent advances, conventional systems suffer from a number of technical deficiencies, particularly with regard to accuracy, efficiency, and operational inflexibility in computer-implemented drug discovery platforms for treating diseases.

### SUMMARY

Embodiments of the present disclosure provide benefits and/or solve one or more of the foregoing or other problems in the art with systems, non-transitory computer-readable media, and methods for signature discovery related to genes correlating with gene expression sensitivity to a target compound/target perturbation. Specifically, the disclosed systems can implement a signature discovery pipeline that applies a target compound to multiple data modalities (e.g., different pre-clinical datasets) to generate sets of gene differential expression measures. Further, the disclosed systems can generate a multi-modal ranked set of genes from sets of gene differential expression measures that are generated from a differential expression model. Moreover, the disclosed systems generate a gene signature for a target compound from the multi-modal ranked set of genes.

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates an overview diagram of a signature discovery system generating a gene signature in accordance with one or more embodiments.

FIG. 2 illustrates an example diagram of the signature discovery system generating area under the curve metrics and using a differential expression model to generate a set of gene differential expression measures in accordance with one or more embodiments.

FIG. 3 illustrates an example diagram of the signature discovery system applying sensitivity weights to a subset of samples to generate gene differential expression measures in accordance with one or more embodiments.

FIG. 4 illustrates an example diagram of the signature discovery system generating a multi-modal ranked set of genes in accordance with one or more embodiments.

FIG. 5 illustrates an example diagram of the signature discovery system performing leave-one-out cross-validation to select a subset of genes in accordance with one or more embodiments.

FIG. 6 illustrates an example diagram of the signature discovery system performing overrepresentation analysis, community detection, and feature selection to generate a gene signature in accordance with one or more embodiments.

FIG. 7 illustrates example results of the signature discovery system generating gene differential expression measures with varying levels of sensitivity to a target compound in accordance with one or more embodiments.

FIG. 8 illustrates an example diagram of the signature discovery system computing a gene signature in accordance with one or more embodiments.

FIG. 9 illustrates the signature discovery system generating gene signatures for causal discovery in accordance with one or more embodiments.

FIG. 10 illustrates example results of the signature discovery system generating gene signatures that outperform existing signatures in accordance with one or more embodiments.

FIG. 11 illustrates an example environment of the signature discovery system in accordance with one or more embodiments.

FIG. 12 illustrates an example series of acts to generate a gene signature in accordance with one or more embodiments.

FIG. 13 illustrates a block diagram of a computing device for implementing one or more embodiments.

### DETAILED DESCRIPTION

Embodiments of the present disclosure provide benefits and/or solve one or more of the foregoing or other problems in the art with systems, non-transitory computer-readable media, and methods for a signature discovery system that utilizes computer-implemented models to generate biomolecule signatures (e.g., gene signatures) whose abundance/expression correlates with sensitivity to a target compound (e.g., or a target gene perturbation). In one or more embodiments, the signature discovery system implements a signature pipeline to transform/account for biological data from multiple sources of information for a potential compound. For example, the signature discovery system can consider a cell line data set treated with a target compound and an organoid data set also treated with the target compound. The signature discovery system can perform differential expression on both data sets, generate gene rankings from each dataset, and combine the rankings for a custom form of differential expression analysis. Specifically, the signature discovery system ranks each dataset according to their log fold change (e.g., log transformed area under the curve metrics obtained from dose response curves), averages the ranks across datasets, and compares the ranks to a null distribution of ranks of genes across both data sets. This allows the signature discovery system to determine significance measures (e.g., p-values) and identify those genes that are consistent between the two data sets.

Moreover, in some embodiments, to account for small sample size and the potential for unduly influential samples, the signature discovery system also performs leave-one-out cross validation by iteratively removing a single sample and analyzing the resulting differentially expressed genes (and using those genes that are not influenced by a single sample).

In generating differential expression measures, the signature discovery system can also weight samples (e.g., utilizing sensitivity weights) depending on high area under the curve metrics or low inhibitory concentration metrics (e.g., IC50 values). Further, in evaluating differential expression measures for ranking, the signature discovery system can apply bias weights to bias the resulting expression measures more towards one dataset (e.g., experimenters may trust the organoid data more than the cell line data because it is closer to the patient). These techniques more accurately identify differentially expressed genes that are then utilized in overrepresentation analysis, community detection, and/or feature selection (maximum relevance minimum redundancy features selection) to identify a list of signatures associated with biological themes (e.g., biological pathways) that correlate with sensitivity to a molecule.

The output from the signature pipeline can then be utilized in a variety of ways, including target identification (e.g., hit-to-lead pipelines), mechanism of action discovery (e.g., lead optimization), pathway analysis, selecting patients that fit a gene signature for a particular compound to include in a clinical trial (e.g., extracting cancer cell lines from cancer patients to analyze whether their genetic makeup matches the identified gene signatures), and causal discovery for patient stratification.

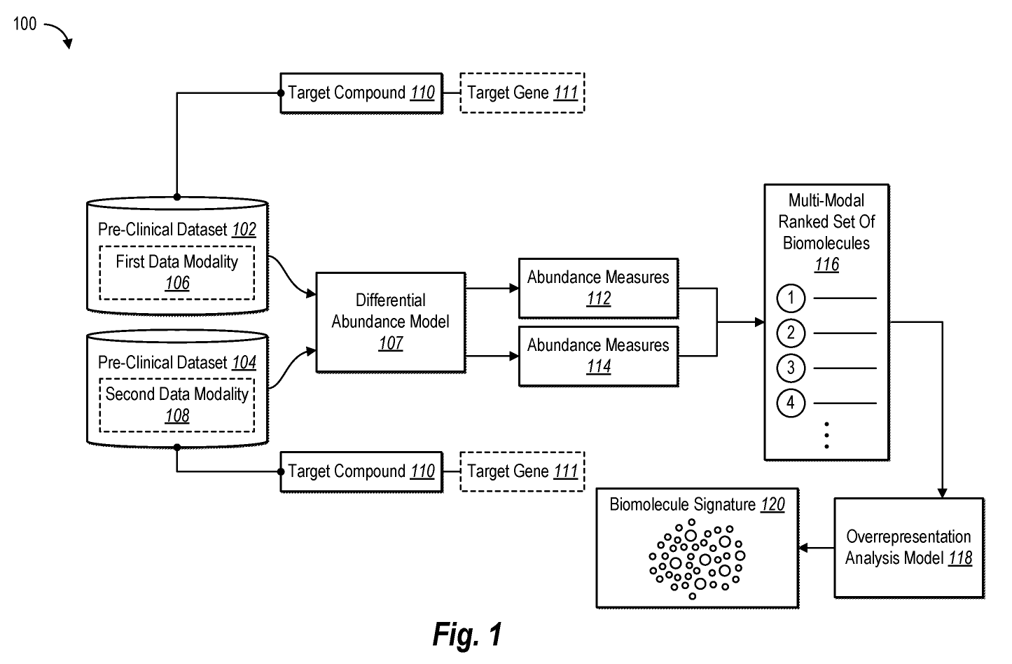

FIG. 1 illustrates a signature discovery system 100 generating a biomolecule signature (e.g., a gene signature) from a multi-modal ranked set of biomolecules in accordance with one or more embodiments. For example, FIG. 1 shows the signature discovery system 100 accessing/receiving pre-clinical datasets relating to different data modalities. As mentioned above, the signature discovery system 100 improves over existing systems by drawing data from multiple different data sources. In particular, FIG. 1 shows the signature discovery system 100 accessing a pre-clinical dataset 102 for a first data modality 106 and a pre-clinical dataset 104 for a second data modality 108. For example, pre-clinical datasets refer to data collected prior to clinical trials (e.g., on humans). Specifically, pre-clinical datasets refer to data generated in laboratory settings that are used to test hypotheses for target validation, identifying candidate therapies, and identifying genes related to biological pathways.

Further, FIG. 1 shows the signature discovery system 100 applying a target compound 110 (e.g., or a target gene 111) to the pre-clinical dataset 102 and the pre-clinical dataset 104. In one or more embodiments, the signature discovery system 100 applies the target compound 110 to a pre-clinical dataset. For example, the target compound 110 refers to a specific chemical compound/substance that interacts with a biological target to achieve a desired biological effect. In other words, the target compound 110 interacts with biological pathways related to a specific disease (e.g., small cell lung cancer). In some embodiments, the target compound 110 activates or inhibits a biological target. In particular, the target compound 110 can inhibit a specific cancer-causing protein or the target compound can cause the upregulation of a specific type of protein.

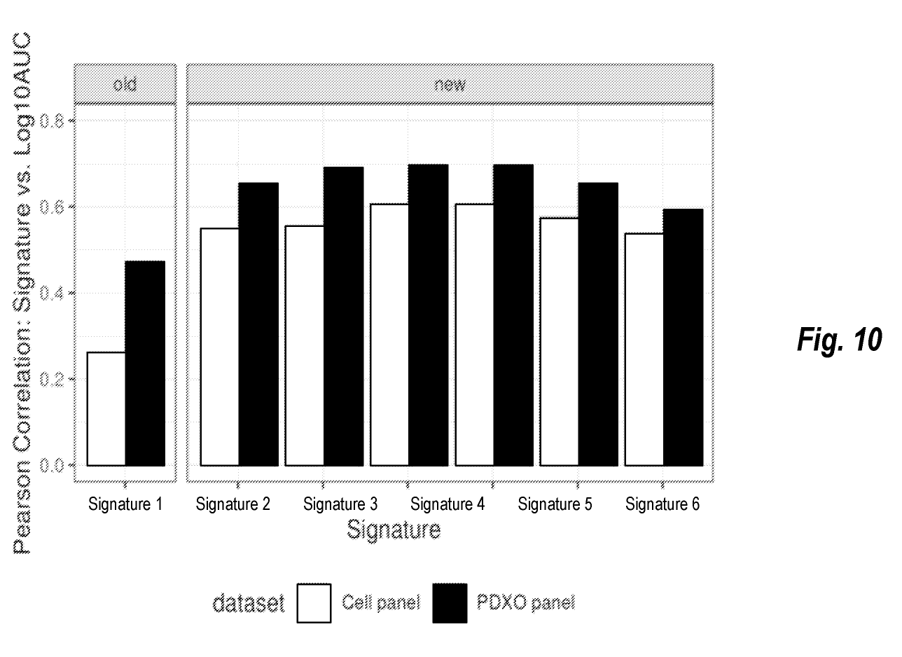

In one or more embodiments, the signature discovery system 100 identifies the target compound 110 from drug development pipelines. In particular, during lead or hit-to-lead stages of drug development, the signature discovery system 100 identifies one or more target compounds to interact with a biological target. Thus, the signature discovery system 100 identifies a disease of interest, a biological pathway associated with the disease of interest and then identifies the target compound 110 that interacts with the biological pathway. Additional details of the signature discovery system 100 utilizing the target gene 111 (e.g., applying a target perturbation to one or more datasets and generating a gene signature) are given below after the description of FIG. 10.

Moreover, FIG. 1 shows the signature discovery system 100 processing the pre-clinical dataset 102 and the pre-clinical dataset 104 (e.g., with the target compound 110 applied) with a differential abundance model 107. In one or more embodiments, the differential abundance model 107 refers to a computer-implemented framework to identify differential abundance from genetic or biological signals. In particular, the differential abundance model 107 can measure multi-omic readouts (e.g., genomics, transcriptomics, proteomics, metabolomics, epigenomics, etc.) from biomolecules (e.g., biological and/or chemical signals).

In some embodiments, the signature discovery system uses the differential abundance model 107 to measure the abundance of biomolecules. In particular, biomolecules refer to a wide variety of biological molecules, such as genes, proteins, RNA, metabolites, lipids, carbohydrates, nucleotides, etc. (e.g., other omic readouts). For example, the signature discovery system 10 utilizes the differential abundance model 107 to measure a difference in the abundance or concentration of specific molecules across different conditions or groups. In other words, the differential abundance model 107 measures a relative or absolute quantity of metabolites, microbial species, or genes without implying the gene or protein is differentially expressed at a transcriptional level (e.g., metabolite abundance being higher in a diseased state relative to a healthy state).

In some embodiments, the signature discovery system 100 utilizes a differential expression model as the differential abundance model 107. In one or more embodiments, the differential expression model refers to a computer-implemented framework to identify genes or biological features whose expression levels vary significantly (e.g., satisfy a significance expression threshold) between different conditions, treatments, or groups. In particular, the signature discovery system 100 uses the differential expression model to identify one or more genes that are differentially expressed under certain experimental conditions/treatments. For example, the signature discovery system 100 applies a target compound (or knockout/perturbation a target gene) to sample data and measures the resulting expression levels. In some embodiments, the signature discovery system 100 applies the target compound 110 (or target perturbation) to a dataset of samples and measures expressions levels such as counts, reads, or log-transformed values.

As shown in FIG. 1, based on using the differential abundance model107, the signature discovery system 100 generates abundance measures 112 and abundance measures 114 (e.g., differential abundance measures). In particular, the signature discovery system 100 generates the abundance measures 112 for the pre-clinical dataset 102 of the first data modality 106 and the abundance measures 114 for the pre-clinical dataset 104 of the second data modality 108.

In some embodiments, the signature discovery system 100 generates differential expression measures as the abundance measures 112 and the abundance measures 114. For example, the gene differential expression measures refer to the upregulation or downregulation of genes in the pre-clinical datasets based on applying the target compound 110 (e.g., or the target perturbation).

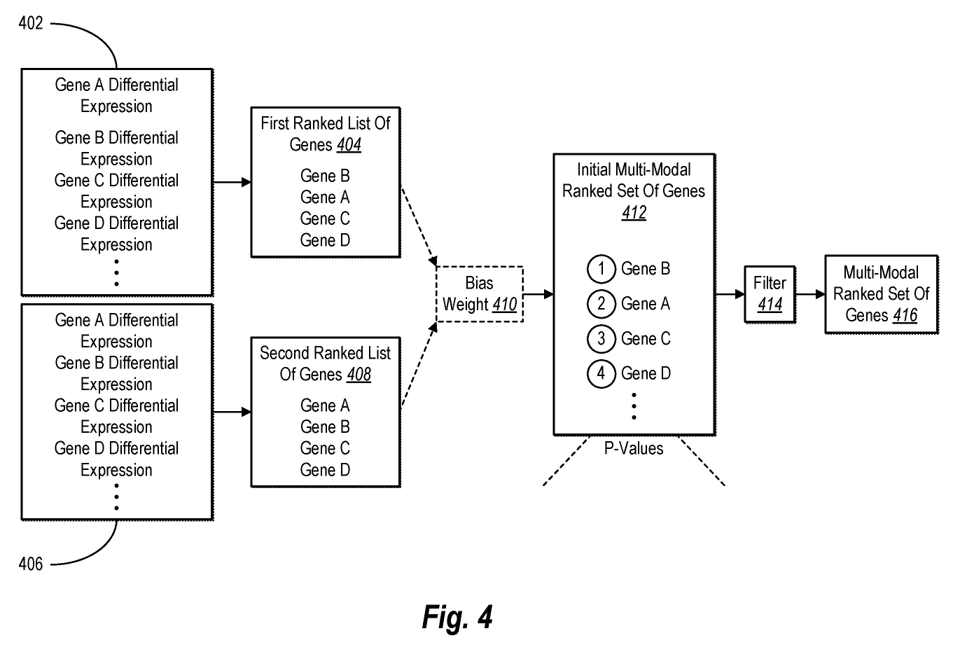

Additionally, FIG. 1 shows the signature discovery system 100 generating a multi-modal ranked set of biomolecules 116. For example, the multi-modal ranked set of biomolecules refers to a list of ranked biomolecules for both the first data modality 106 and the second data modality 108. In some embodiments, the signature discovery system 100 generates a multi-modal ranked set of genes. To illustrate, the signature discovery system 100 can determine RNA expression metrics (such as area under the curve) corresponding to different perturbations and different modalities. The signature discovery system 100 utilize a differential expression model to generate differential expression measures and ranked sets of genes for the different modalities. The system then combines the ranked sets of genes to generate a multi-modal ranked set of genes. Additional details of generating the multi-modal ranked set of genes is given below in the description of FIG. 4.

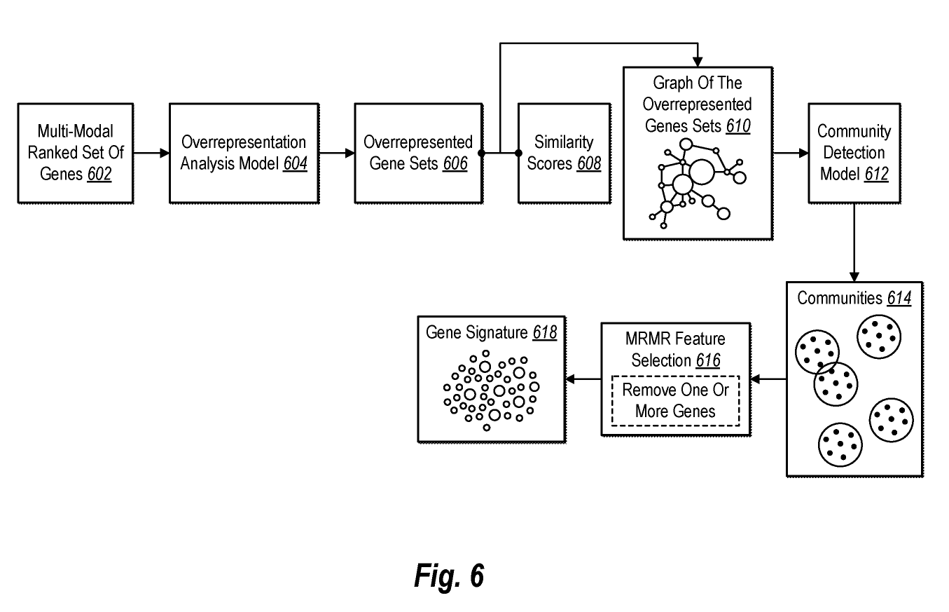

Moreover, FIG. 1 shows the signature discovery system 100 using an overrepresentation analysis model 118 on the multi-modal ranked set of biomolecules 116 to generate a biomolecule signature 120. In particular, the biomolecule signature 120 refers to related biomolecules that correspond to biological pathways. Indeed, the signature discovery system 100 can utilize the overrepresentation analysis model 118 to identify overrepresented biomolecules in the multi-modal ranked set of biomolecules. Moreover, the signature discovery system can utilize a community detection model to identify communities and corresponding biological pathways. In this manner, the signature discovery system can generate biomolecule signatures that correspond to biological pathways (instead of random gene clusters or groups). Additional details of using the overrepresentation analysis model 118 is given below in the description of FIG. 6.

In some embodiments, the signature discovery system 100 generates the gene differential expression measures, the multi-modal ranked set of genes, and further generates a gene signature as the biomolecule signature 120. In one or more embodiments, the signature discovery system 100 generates the gene signature for the target compound 110. In particular, the gene signature includes a subset of genes from the multi-modal ranked set of genes that correlate with gene expression sensitivity to the target compound 110. For example, the signature discovery system 100 generates the gene signature to identify a relevant subset of genes for a target compound, where the relevant subset of genes indicates a threshold level of sensitivity to inhibiting a target gene by utilizing the target compound.

In other words, the signature discovery system 100 more effectively treats a specific disease with a target compound by generating a gene signature. In some embodiments, the signature discovery system 100 extracts patient samples (e.g., such as cancer cells from a patient) and determines whether the patient samples include the generated gene signature. If the patient sample does include the generated gene signature, then the signature discovery system 100 determines that the target compound has a high likelihood of effectively targeting a specific disease (e.g., biological pathway corresponding to the specific disease) in the patient.

As mentioned briefly above, existing systems suffer from inaccuracies, inefficiencies, and operational inflexibilities. Many existing systems perform drug development/analysis in a complex and protracted manner that involves several technical steps. In particular, existing systems typically analyze a large volume of gene expression data from a single data modality to attempt to identify important genes for a biological objective. Moreover, from analyzing the large volume of gene expression data, existing systems then identify genes. In one or more embodiments, existing systems can inaccurately identify genes for a biological objective. For example, conventional systems cannot strongly predict sensitivity to compounds within cancer subtypes across preclinical models. Moreover, existing systems identify signatures that are not well expressed in patient samples, that suffer from technical artifacts, and that are sensitive to small sample sizes. Furthermore, gene signatures generated by conventional systems often have no correlation to relevant biology but represent (often irrelevant) combinations of different genes. In other words, existing systems are unable to work with multiple data modalities in analyzing genes for a biological objective. As such, existing systems inaccurately identify genes for a biological objective.

Existing systems suffer from inaccuracies and can require tens of thousands of iterations to identify genes that are sensitive to a pharmaceutical compound viable for therapeutic treatment. Indeed, existing systems may inaccurately parse through a large volume of genes to identify a set of genes that may interact with a target within a threshold amount. The sheer volume of data required for existing systems to parse through presents accuracy challenges in itself, as existing systems fail to filter down a large volume of data in a manageable and accurate manner.

Moreover, existing systems tend to output a high number of false positives and false negatives. In particular, existing systems tend to identify sets of genes that appear effective in interacting with a target compound but are not actually effective. Further, existing systems often overlook sets of genes that could have been effective in being sensitive to a target compound. Moreover, existing systems often generate false positives and false negatives as they are restricted to working with a single data modality and are unable to cross-verify/validate the veracity of their data. As a result, existing systems waste resources on false positives and false negatives, and cause inefficiencies in the overall process. Accordingly, existing systems typically iterate through an excessive number of rounds of analyzing a large volume of genes and filtering down a large volume of genes to arrive at a set of genes that is believed to correlate with gene expression sensitivity to a compound (e.g., however they are often inaccurate).

Moreover, existing systems suffer from operational inflexibilities due to being constrained to the inefficient and inaccurate drug development/analysis process described above. In one or more embodiments, existing systems are rigidly confined to existing methods to measure the gene expression of genes from applying a target compound. In particular, existing methods may not always accurately measure the impact of a target compound on genes and further fail to efficiently sort through a large volume of data. In addition, existing systems are unable to flexibly adapt performance to different preclinical models or modalities that represent patient samples. Thus, the operational constraints of existing drug design processes to identify sets of genes further exacerbate efficiency and accuracy concerns.

In one or more embodiments, the signature discovery system 100 improves accuracy, efficiency, and operational flexibility relative to existing systems. As mentioned above, existing systems typically analyze a large volume of gene expression data from a single data modality to attempt to identify important genes for a biological objective. In contrast, the signature discovery system 100 accesses different data modality datasets (e.g., cell line dataset and an organoid dataset), generates gene differential expression measures for each of the different data modality datasets, and further generates a multi-modal ranked set of genes for the different data modality datasets. In doing so, the signature discovery system 100 can concurrently analyze different data modalities and identify genes consistent across the different data modalities. Accordingly, the signature discovery system 100 can generate a gene signature (e.g., a subset of genes) that more accurately correspond with gene expression sensitivity to a target compound (e.g., relative to existing systems).

Additionally, the signature discovery system 100 improves accuracy by finding/identifying a subset of genes that are consistent between the different data modality datasets. In particular, the signature discovery system 100 is able to find genes that are consistently differentially expressed across different data modality datasets. In other words, in generating gene signatures, the signature discovery system 100 also enhances the confidence of the subset of genes correlating with gene expression sensitivity to a target compound, as the identified subset of genes are consistently expressed across different data modality datasets. Moreover, the signature discovery system 100 can strongly predict sensitivity to compounds across and within disease cell subtypes in preclinical models. In addition, the signature discovery system can generate gene signatures that are well expressed in patient samples, that are robust to technical artifacts (e.g., batch effects, sample purity, etc.), and that are robust to small sample size. Moreover, the signature discovery system can generate gene signatures that represent relevant biology (e.g., a collection of related genes that relate to compound biology, rather than randomly selected disparate gene groups).

Moreover, in some embodiments, the signature discovery system 100 further improves upon accuracy relative to existing systems by establishing a signature pipeline that uses a custom form of differential expression analysis (e.g., AUC, IC50, bias weights, and sensitivity weights) to transform biological signals. Specifically, the signature discovery system 100 uses a differential expression model with area under the curve metrics of dose response curves for a target compound applied to a dataset of genes. For example, the signature discovery system 100 feeds as input the area under the curve metrics to a differential expression model to generate gene differential expression measures and to further generate a multi-modal ranked set of genes. In other words, the custom form of differential expression analysis allows the signature discovery system 100 to more accurately home in on genes that are most sensitive to a target compound.

Furthermore, in some embodiments, the signature discovery system 100 improves upon accuracy of customizing/tailoring drug treatment of patients. In one or more embodiments, the signature discovery system 100 generates a gene signature that correlates with gene expression sensitivity to a target compound. Moreover, in some embodiments, the signature discovery system 100 accesses extracted biological data from a cancer patient (e.g., cancer cell lines), analyzes the extracted biological data, and determines whether the cancer patient possesses the generated gene signature. In other words, the signature discovery system 100 can identify cancer patients that would most effectively respond to target compound treatment based on the generated gene signature.

Additionally, in some embodiments, the signature discovery system 100 improves upon accuracy of identifying similar gene sets or drug compounds. Specifically, the signature discovery system 100 can improve upon the hit-to-lead process or the lead optimization process of developing related drugs for treating related diseases. In one or more embodiments, the signature discovery system 100 generates the gene signature that correlates with sensitivity to a target compound and then further uses that data to cross-reference overlapping biological pathways/substrates and initiates further drug development processes. In other words, the signature discovery system 100 aids drug discovery pipelines to identify overlapping use cases with the target compound.

As mentioned above, existing systems output a high number of false positives and false negatives. In contrast, the signature discovery system 100 improves efficiency by more effectively transforming biological signals (e.g., data from pre-clinical datasets), analyzing the data, and generating a gene signature. In particular, the signature discovery system 100 uses a custom form of differential expression analysis which more effectively outputs genetic expression measures for analysis and ranking. Accordingly, the signature discovery system 100 reduces the number of iterations and the effectiveness of parsing down a large volume of data to the gene signature that signifies gene expression sensitivity to a target compound. In other words, the signature discovery system 100 more effectively identifies sets/subsets of genes that are sensitive to a target compound relative to existing systems.

Relatedly, the signature discovery system 100 improves upon operational flexibility. As mentioned above, the signature discovery system 100 is not constrained to static differential expression methods but rather implements a custom form of differential expression to analyze, rank, and generate gene signatures. Moreover, the signature discovery system 100 can adjust performance to emphasize preclinical models that represent patient samples. Indeed, the signature discovery system 100 can utilize a flexible weighting approach in applying computer-implemented models to achieve strongest performance in models that best represent patient samples. Thus, the signature discovery system 100 more accurately and effectively generates a gene signature.

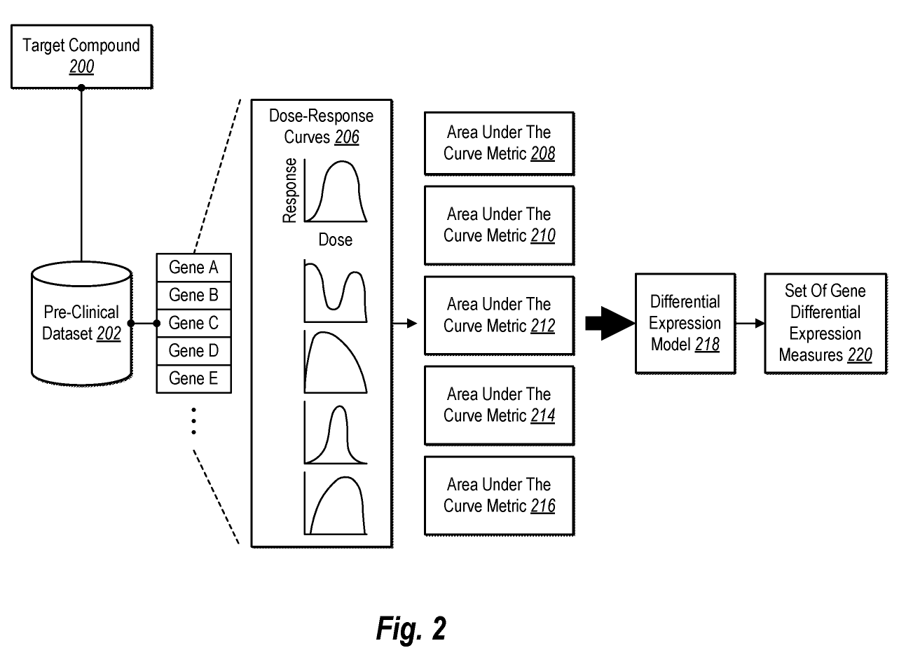

FIG. 2 illustrates the signature discovery system 100 using a custom form of differential expression by generating area under the curve metrics in accordance with one or more embodiments. For example, FIG. 2 shows the signature discovery system 100 accessing/receiving a pre-clinical dataset 202 with a target compound 200 applied to the pre-clinical dataset 202. In particular, FIG. 2 shows that the pre-clinical dataset 202 corresponds to a plurality of genes (e.g., genes A-E…).

Moreover, FIG. 2 shows the signature discovery system 100 applying different doses of the target compound 200 to each sample of the pre-clinical dataset 202. In particular, FIG. 2 shows dose-response curves 206 (e.g., for each sample of the pre-clinical dataset 202). For example, the signature discovery system 100 generates area under the curve metrics by plotting dose response curves for the sample data with the target compound 200 applied. In particular, a dose response curve refers curve indicating a relationship between the dose (e.g., of the target compound) and the magnitude of the biological response (e.g., expression) that the dose produces.

In some embodiments, the dose refers to the concentration of the target compound 200 administered to the dataset samples (e.g., plotted on the x-axis). In particular, the signature discovery system 100 uses a dose on a logarithmic scale (log₁₀) because biological systems often show a wide range of responses to different concentrations of a target compound. Moreover, in some embodiments, the response refers to the observed effect of the target compound at a specific dose (e.g., plotted on the y-axis).

In one or more embodiments, a dose response curve has a threshold where at very low doses, there is no significant response to the dose. Further, in some embodiments, as the dose increases, the response can increase in a linear or a log fashion. For different samples, the response to a target compound varies drastically and is not typically a linear response.

In one or more embodiments, the signature discovery system 100 generates dose response curves for each sample of a dataset of samples. In particular, the signature discovery system 100 utilizes a dose response curve to determine how sensitive and potent a sample is to a target compound. In some embodiments, the signature discovery system 100 determines an IC50 (e.g., inhibitory concentration 50) from the dose response curve.

In some embodiments, the IC50 refers to a dose of the target compound applied to a sample at which 50% of the maximum inhibition is achieved. In other words, the signature discovery system 100 determines the IC50 as a measure to assess the potency of the target compound in inhibiting a particular genes (e.g., biological or biochemical function). In particular, the IC50 represents a concentration of an inhibitor (e.g., the target compound 200) required to reduce the biological response of a sample (e.g., the gene) by 50%. To illustrate, for a dose response curve (e.g., such as a sigmoidal S-shaped curve), the signature discovery system 100 determines a concentration of the target compound where the response is halfway between a baseline (e.g., no inhibition) and the maximal inhibition. In other words, the signature discovery system 100 determines 50% of the maximum possible inhibition.

As shown in FIG. 2, the signature discovery system 100 generates an area under the curve metric 208, an area under the curve metric 210, an area under the curve metric 212, an area under the curve metric 214, and an area under the curve metric 216. In one or more embodiments, the signature discovery system 100 generates area under the curve metrics from dose-response curves. In particular, the signature discovery system 100 generates a first area under the curve metric for a first dose-response curve corresponding to a first sample, a second area under the curve metric for a second dose-response curve corresponding to a second sample, etc.

The signature discovery system 100 can quantify a total effect or response of a target compound applied to a sample over a range of doses. In one or more embodiments, the signature discovery system 100 determines a dose range (e.g., minimum dose to maximum dose) and calculates the area under the dose-response curve for the determined range. In particular, the signature discovery system 100 generates an area under the curve metric using numerical integration methods such as a trapezoidal calculation (e.g., approximate the area under the curve by dividing the total rea into a series of trapezoids). To illustrate, a larger area under the curve metric indicates a greater total biological effect of applying the target compound to a sample over the dose range. In contrast, a smaller area under the curve metric indicates a less potent or effective substance, such as a drug that requires a higher dose to achieve a similar effect.

In one or more embodiments, the signature discovery system 100 uses Limma-Voom differential expression that performs regression (e.g., linear regression) on the data using log₁₀ area under the curve measurements. To illustrate, the signature discovery system 100 uses a Limma-Voom differential expression pipeline as a differential expression model 218. In particular, the signature discovery system 100 uses Limma-Voom to identify genes up or down-regulated in sensitive versus resistant models. In other words, the signature discovery system 100 establishes sensitive models for samples that are sensitive to a specified disease (e.g., respond to treatment), and for samples that are resistant to a specified disease (e.g., samples that do not respond to treatment). Moreover, the signature discovery system 100 compares the various modes of data with the target compound applied to the sensitive and resistant models to determine measures of differential expression.

In one or more embodiments, the signature discovery system 100 uses the Limma-Voom differential expression model which uses linear modeling techniques for RNA-sequencing data. In particular, the signature discovery system 100 uses the Limma-Voom differential expression model which uses a variance-stabilizing transformation to make the data more suitable for analysis. For example, the signature discovery system 100 uses the Limma-Voom differential expression model to perform data transformations on the area under the curve metrics (or other expression metrics), such as calculating the variance or mean of each AUC metric for a gene between data modalities.

Moreover, in some embodiments, the signature discovery system 100 uses the Limma-Voom differential expression model to fit a linear model to the transformed data to test for differential expression. In particular, the signature discovery system 100 can use the Limma-Voom differential expression model to output a set of gene differential expression measures 220. In some embodiments, the signature discovery system 100 determines whether a gene is differentially expressed based on comparing the data (e.g., with the target compound applied to the data) to various statistical significance tests (e.g., data points corresponding to genes that satisfy a statistical significance threshold is considered differentially expressed).

The signature discovery system 100 can utilize a variety of differential expression models. For example, the signature discovery system 100 can utilize Limma-Voom, DESeq2, edgeR, baySeq, NOISeq, SAMseq, Sleuth, or other differential expression models.

In some embodiments, the signature discovery system 100 performs a log fold-change test (a size of the difference in AUC metrics between conditions) between conditions, such as how much more or less AUC metrics are in a low dose compared to a control, a p-value test (e.g., probability that an observe difference of the AUC metric compared to a control could have occurred by chance), an adjusted p-value test of the AUC metrics compared to a control (e.g., correcting for multiple test to control for false discovery rate), or a B-statistic test (a measure of the strength of evidence of differential expression).

As shown, the signature discovery system 100 uses the differential expression model 218 to generate the set of gene differential expression measures 220. In one or more embodiments, the signature discovery system 100 generates differential expression measures for the pre-clinical dataset 202. In particular, the signature discovery system 100 generates the set of gene differential expression measures 220 which indicate which genes are differentially expressed for certain conditions (e.g., applying the target compound 300 at a certain dose). For example, the signature discovery system 100 can use pre-clinical datasets for various data modalities such as cell line data and organoid data, which is discussed in more detail below.

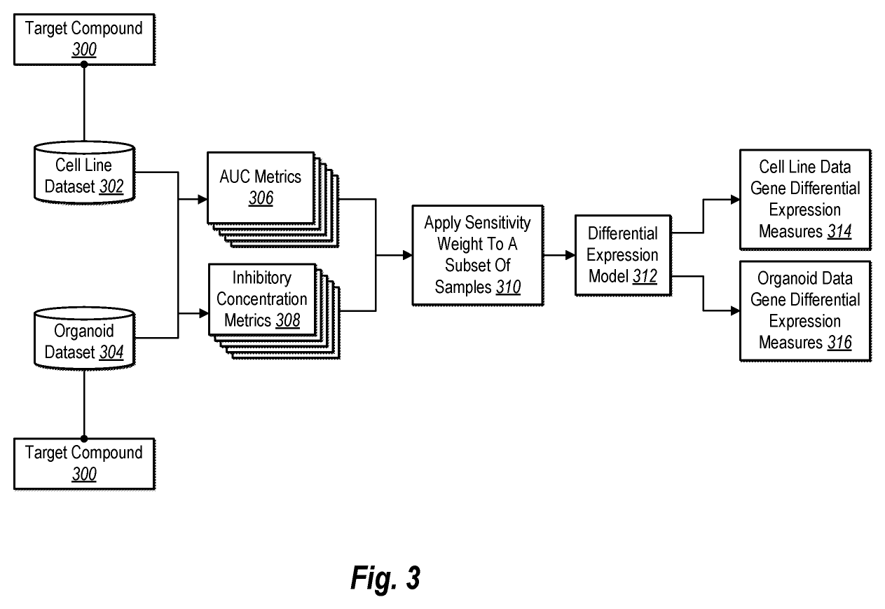

As shown in FIG. 3, the signature discovery system 100 accesses different pre-clinical datasets relating to different data modalities and generates gene differential expression measures in accordance with one or more embodiments. For example, FIG. 3 shows the signature discovery system 100 accessing a cell line dataset 302 and an organoid dataset 304 with the target compound 300 applied. In particular, the signature discovery system 100 accesses the different data modalities and generates gene differential expression measures.

In one or more embodiments, the signature discovery system 100 generates gene differential expression measures from the cell line dataset 302 (e.g., a first or second pre-clinical dataset that relates to a specific data modality of cell lines) that includes expression data from applying the target compound 300 to a plurality of cells from a plurality of cell lines. In particular, the cell line dataset 302 refers to a population of cells that originates from a single ancestral cell. For example, cell lines can include human cell lines (e.g., derived from human tissue), animal cell lines (e.g., derived from non-human species), and cancer cell lines (e.g., derived from tumor tissue).

In one or more embodiments, the signature discovery system 100 generates gene differential expression measures from the organoid dataset 304 (e.g., a first or second pre-clinical dataset for a specific data modality of organoid data). For example, the organoid dataset 304 refers to three-dimensional (3D) cell culture models that mimic the structure, function, and complexity of real organs. In particular, organoids refer to stem cells that replicate key features of an organ. In particular, the signature discovery system 100 generates gene differential expression measures from the organoid dataset 304 that includes expression data from applying the target compound to a plurality of xenograft organoids.

In some embodiments, a xenograft organoid refers to a laboratory grown, 3D model that combines organoid culture systems with a xenograft approach by implanting patient-derived tumor tissue into an animal to mimic a human tumor microenvironment. Thus, the signature discovery system 100 applies the target compound 300 to the organoid dataset 304 and generates measures of gene differential expression for a plurality of genes.

In one or more embodiments, the signature discovery system 100 generates gene differential expression measures for a plurality of genes in relation to sensitivity from applying the target compound 300 to the pre-clinical dataset (e.g., the cell line dataset 302 or the organoid dataset 304). For example, a gene refers to segments of deoxyribonucleic acid (DNA) that are responsible for coding instructions to produce proteins and regulate biological functions within an organism. In particular, a gene includes a specific sequence of nucleotides (e.g., adenine, thymine, cytosine, and guanine) and the sequence (order) of the nucleotides determines the function of the gene (e.g., how it makes proteins). Thus, the signature discovery system 100 applies the target compound to the pre-clinical datasets and generates measures of gene differential expression for a plurality of genes.

FIG. 3 shows the signature discovery system 100 generating AUC metrics 306 and inhibitory concentration metrics 308 from applying the target compound 300 to the cell line dataset 302 and the organoid dataset 304. Moreover, FIG. 3 shows the signature discovery system 100 performing an act 310 of applying sensitivity weights to a subset of samples from the cell line dataset 302 and/or the organoid dataset 304.

In one or more embodiments, the signature discovery system 100 utilizes sensitivity weights to generate differential expression measures. In particular, the signature discovery system 100 biases differential expression results to more sensitive samples (e.g., that indicate especially high area under the curve metrics and low inhibitory concentration metrics, IC50). For example, the signature discovery system 100 generates the area under the curve metrics for a plurality of samples (e.g., corresponding to a plurality of genes in a dataset), identifies a subset of the samples that satisfy/exceed a threshold value for area under the curve metrics, and applies sensitivity weights to the subset of the samples. In other words, the signature discovery system 100 applies the sensitivity weights to inflate the differential expression results for the identified subset of samples.

Additionally, as shown in FIG. 3, the signature discovery system 100 uses a differential expression model 312 to process the AUC metrics 306 (e.g., with the sensitivity weights applied based on the AUC metrics 306 and/or the inhibitory concentration metrics 308). Further, FIG. 3 shows the signature discovery system 100 generating cell line data gene differential expression measures 314 and organoid data gene differential expression measures 316 from using the differential expression model 312.

FIG. 4 illustrates the signature discovery system 100 generating a multi-modal ranked set of genes in accordance with one or more embodiments. For example, FIG. 4 shows the signature discovery system 100 generating gene differential expression measures for each data modality. In particular, similar to FIG. 3, FIG. 4 shows a first set of gene differential expression measures 402 for a first data modality and a second set of gene differential expression measures 406 for a second data modality (e.g., the gene differential expression measures include various metrics (log transformed values) of each gene from each data modality).

Furthermore, FIG. 4 shows the signature discovery system 100 generating a first ranked list of genes 404 and a second ranked list of genes 408 for the first set of gene differential expression measures 402 and for the second set of gene differential expression measures 406. In one or more embodiments, the signature discovery system 100 generates a ranked list of genes for each data source (e.g., pre-clinical dataset). In some embodiments, the signature discovery system 100 generates a ranked list of genes according to the measures of gene differential expression.

In one or more embodiments, the signature discovery system 100 generates the gene differential expression measures based on the AUC metrics. In particular, the signature discovery system 100 generates the AUC metrics and applies a log transformation (e.g., a log fold change such as log₁₀) to the metrics using the differential expression model. In doing so, the signature discovery system 100 normalizes the data to reduce skewness, stabilize variance, and make patterns in the data more linear. Moreover, in some embodiments, from applying the log transformation, the signature discovery system 100 uses the differential expression model to generate gene differential expression measures and ranks them according to descending log transformed metrics (from greatest to least).

As shown, in one or more embodiments, the signature discovery system 100 further applies a bias weight 410 to the first ranked list of genes 404 or to the second ranked list of genes 408. For example, the signature discovery system 100 applies the bias weight 410 that weights samples for a specific pre-clinical dataset greater than other pre-clinical datasets. In other words, the signature discovery system 100 biases the rankings towards a more clinically relevant pre-clinical dataset. In some embodiments, the signature discovery system 100 applies the bias weight 410 to the organoid data such that the organoid data is weighted greater (e.g., 2:1 weighted or 9:1 weighted) than the cell line data.

As further shown, the signature discovery system 100 generates an initial multi-modal ranked set of genes 412 (e.g., an initial multi-modal ranked list). In one or more embodiments, the signature discovery system 100 combines the first ranked list of genes 404 and the second ranked list of genes 408 to generate the initial multi-modal ranked set of genes 412. In particular, the signature discovery system 100 averages the ranks of the first ranked list of genes 404 and the second ranked list of genes 408.

In other words, the signature discovery system 100 combines the first ranked list of genes 404 and the second ranked list of genes 408 in such a manner to reflect the relative importance/significance of each gene across each data modality (e.g., cell line data and organoid data). For instance, the signature discovery system 100 takes the gene differential expression measure for gene A from the cell line data and the gene differential expression measure for gene A from the organoid data and generates a combined score (e.g., an average gene differential expression measure for gene A). Thus, in some embodiments, the signature discovery system 100 further generates the initial multi-modal ranked set of genes 412 from the averaged measures (e.g., combined score) for each gene. The signature discovery system 100 can utilize a variety of approaches to combining the ranked set of genes, including averaging, adding, weighted averaging, or another combination technique.

Moreover, as shown in FIG. 4, in some embodiments the signature discovery system 100 further performs an act 414 of filtering one or more genes from the initial multi-modal ranked set of genes 412. In particular, the signature discovery system 100 filters the initial multi-modal ranked set of genes 412 based on p-values.

In some embodiments, the signature discovery system 100 generates statistical significance measures for the initial multi-modal ranked set of genes 412. For example, the signature discovery system 100 generates p-values for the initial multi-modal ranked set of genes 412 that indicates a significance of a gene in context of a hypothesis test. In particular, the signature discovery system 100 uses a null hypothesis that represents the idea that the target compound has no effect or no difference on the gene expression of a particular gene.

In other words, the p-value quantifies the significance of the gene expression of the gene when the target compound is applied. In one or more embodiments, the signature discovery system 100 generates the gene differential expression measures for the plurality of genes and compares the gene differential expression measures to a null distribution of ranks. In particular, a null distribution of ranks represents an expected behavior of a test statistic or outcome under the null hypothesis. In other words, the null distribution represents a distribution of gene expression measures if there were no real effect or no relationship in the data from applying the target compound to a dataset (e.g., the null hypothesis is true).

As shown in FIG. 4, from performing the act 414 of filtering the initial multi-modal ranked set of genes 412, the signature discovery system 100 generates a multi-modal ranked set of genes 416. In particular, the signature discovery system 100 filters one or more genes from the initial multi-modal ranked set of genes 412 using the statistical significance measures. For example, the signature discovery system 100 establishes a threshold (e.g., p value less than or equal to 0.05) and removes genes from the initial multi-modal ranked set of genes 412 that fail to satisfy the threshold to generate the multi-modal ranked set of genes 416.

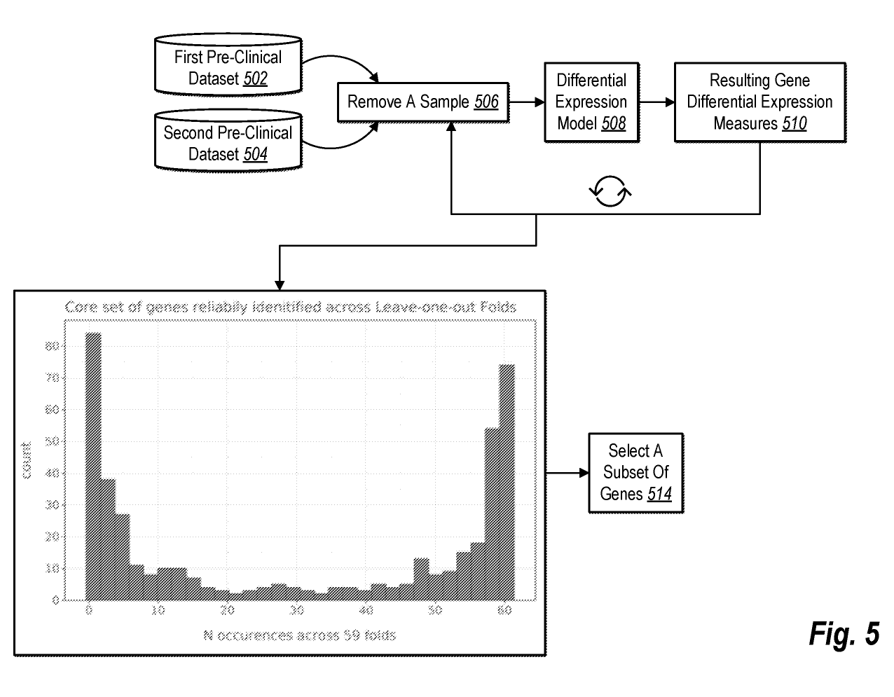

FIG. 5 illustrates the signature discovery system 100 performing leave-one-out cross-validation to iteratively remove individual samples and generating gene differential expression measures in accordance with one or more embodiments. For example, FIG. 5 shows the signature discovery system 100 accessing a first pre-clinical dataset 502 and a second pre-clinical dataset 504 and performing an act 506 of removing a sample. In particular, as shown, after removing a sample from either the first pre-clinical dataset 502 or the second pre-clinical dataset, the signature discovery system 100 uses a differential expression model 508 to generate a resulting gene differential expression measures 510.

Moreover, FIG. 5 shows the signature discovery system 100 iteratively repeating the process shown to remove a different individual sample from the first pre-clinical dataset 502 or the second pre-clinical dataset 504 each round. Thus, the signature discovery system 100 generates resulting gene differential expression measures for each round of removing an individual sample from the first pre-clinical dataset 502 or the second pre-clinical dataset 504.

In one or more embodiments, the signature discovery system 100 performs leave-one-out cross-validation for differential expression analysis to filter out samples (e.g., genes) where a data source (e.g., cell line) is overly contributing to differential expression. For example, the signature discovery system 100 performs leave-one-out cross-validation by removing a different individual sample each round of differential expression analysis. In particular, the signature discovery system 100 iteratively removes individual samples from data sources (e.g., the first pre-clinical dataset or the second pre-clinical dataset) and compares resulting gene differential expression measures.

To illustrate, the signature discovery system 100 performs leave-one-out cross-validation to identify groups of genes that robust to small changes in the data by leaving out a single sample, performing differential expression analysis, generating differential expression measures, and then removing a different sample and re-performing differential expression analysis.

In one or more embodiments, a challenge/concern for relatively small sample sizes of two datasets is identifying gene signatures where the addition or removal of a single sample (e.g., from the first pre-clinical dataset 502 or the second pre-clinical dataset 504) results in greatly altered gene signatures. To combat this, the signature discovery system 100 performs differential expression analysis multiple times (e.g., 59 times or folds, based on the cell line data containing 39 samples and the organoid data containing 19 samples, and one of the folds having no samples left out), each time removing one sample from the first pre-clinical dataset 502 or the second pre-clinical dataset 504 (e.g., cell line data or organoid data).

In one or more embodiments, the signature discovery system 100 performs the leave-one-out cross-validation to eliminate the inclusion of genes whose differential expression was not robust to the removal of individual samples. As shown in FIG. 5, the signature discovery system 100 can generate the graphs the shows resulting gene differential expression measures 510. In particular, the graph shows some genes are not resistant to individual sample removals while some are resistant to individual sample removal. For example, the graph in FIG. 5 shows an x-axis of N occurrences across 59 folds and the y-axis shows counts. In particular, the graph in FIG. 5 shows that at the left side of the distribution, there are genes that are differentially expressed for only a small number of the total 59 folds, while the right side shows some genes that are differentially expressed for most or all of the total 59 folds.

As such, the signature discovery system 100 performs an act 514 of selecting a subset of genes (e.g., the genes that are robust to the removal of individual samples) for ranking. In other words, the signature discovery system 100 performs leave-one-out cross-validation to identify a core set of genes that are reliably identified across leave-one-out folds (e.g., iteratively removing an individual sample and calculating resulting gene differential expression measures).

FIG. 6 illustrates the signature discovery system 100 further performing overrepresentation analysis, community detection, and feature selection to generate a gene signature in accordance with one or more embodiments. For example, FIG. 6 shows the signature discovery system 100 uses an overrepresentation analysis model 604 on a multi-modal ranked set of genes 602.

In one or more embodiments, the signature discovery system 100 uses the overrepresentation analysis model 604 to identify overrepresented gene sets 606 from the multi-modal ranked set of genes 602. For example, the signature discovery system 100 compares the genes in the multi-modal ranked set of genes 602 with a population of genes corresponding to biological pathways to identify the overrepresented gene sets 606. In particular, the signature discovery system 100 compares the multi-modal ranked set of genes 602 with the population of genes (predefined sets of genes that are background genes) from which the multi-modal ranked set of genes 602 were obtained. For instance, the population of genes includes all the genes in a genome or relevant gene list for an organism and the signature discovery system 100 compares the multi-modal ranked set of genes 602 to the population of genes based on different biological pathways (e.g., biological themes).

In one or more embodiments, the signature discovery system 100 performs the comparison to determine whether a specific biological theme is overrepresented in the multi-modal ranked set of genes 602 compared to what would be expected by chance. In particular, the signature discovery system 100 can use a Fisher’s exact test to calculate probabilities of observing the genes in the multi-modal ranked set of genes 602 for a particular biological pathway (e.g., theme) under the assumption that there is no association between the genes and that biological pathway.

Moreover, the signature discovery system 100 generates p-values for each gene for each biological pathway to determine the likelihood of obtaining that gene by random chance (e.g., for the biological pathway). In particular, small p-values indicate that the biological pathway is overrepresented in the multi-modal ranked set of genes 602 (e.g., significantly more likely to occur than by chance) and a large p-value indicates that the biological pathway is not enriched or overrepresented in the multi-modal ranked set of genes 602. Accordingly, the signature discovery system 100 generates the overrepresented gene sets 606, where each overrepresented gene set corresponds to a specific biological pathway.

In one or more embodiments, a biological pathway relates to a series of interconnected molecular interactions and processes that leads to a specific biological outcome. For example, a biological pathway includes molecules such as proteins, lipids, nucleic acids, etc. that interact with each other. In particular, a biological pathway includes signal transduction, enzymatic reactions, activation or inhibition of proteins and other molecules. To illustrate, a biological pathway includes genes regulated by H3K4me2 chromatin marks, genes regulating Epithelial-to-Mesenchymal transition (EMT), Sarcoma progenitors, Lymphoma and leukemia, solute transporters, neurotransmitter release, TP53, APC, MYC and IL2 targets, cancer H3K27me3 targets, apoptosis, insulin signaling pathway, etc. Thus, the signature discovery system 100 determines which biological pathways to analyze against the multi-modal ranked set of genes 602 based on a disease of interest (e.g., small cell lung cancer).

In one or more embodiments, the signature discovery system 100 further generates similarity scores 608 between the overrepresented gene sets 606 for the biological pathways. In other words, the signature discovery system 100 determines the similarity scores 608 between different groups of genes. In particular, the signature discovery system 100 generates a Jaccard similarity between a first overrepresented gene set and a second overrepresented gene set to quantify a level of overlap. For example, the signature discovery system 100 uses a Jaccard index for the two sets (e.g., first overrepresented gene set and the second overrepresented gene set) that determines a size of an intersection of the set (e.g., number of elements common to both sets) and a size of the union of the two sets (the total number of distinct elements in either set). To illustrate a Jaccard similarity of 1 indicates that the sets are identical while a Jaccard similarity of 0 indicates that the gene sets have no common elements.

In one or more embodiments, the signature discovery system 100 generates a graph 610 for the overrepresented gene sets 606. In particular, the signature discovery system 100 generates the graph 610 that includes vertices (nodes) and edges connecting the vertices. For example, the vertices represent the overrepresented gene sets 606 and the edges indicate the calculated similarity scores (e.g. the similarity scores act as a weight between overrepresented gene sets).

As shown in FIG. 6, the signature discovery system 100 further utilizes a community detection model 612 to generate communities 614. In one or more embodiments, the signature discovery system 100 uses the community detection model 612 to generate a community of similar gene sets from the graph 610 of overrepresented genes. For example, the signature discovery system 100 uses the community detection model 612 to identify a partition of the graph that maximizes a modularity score (e.g., a strength of division of a network into communities, where a higher modularity indicates a stronger community structure, many edges within communities and few edges between communities).

In one or more embodiments, the signature discovery system 100 generates a community of similar gene sets from the graph 610 by using the community detection model 612. In some embodiments, a community of similar genes refers to a group of vertices that are more densely connected with each other than with the rest of the graph network (e.g., genes that interact with each other more frequently than other genes within a threshold). To illustrate, the signature discovery system 100 uses Leiden Community Detection which includes an algorithm that processes the graph 610 (e.g., removes vertices with similarity levels that fail to satisfy an established threshold, normalizes edge weights, etc.), and then optimizes the modularity of the graph 610. In particular, optimizing the modularity includes the signature discovery system 100 using Leiden Community Detection to iteratively move nodes between communities (e.g., local refinement), global optimization to further optimize how the graph 610 is divided, and final community extraction to produce a division of the graph 610 into communities.

The signature discovery system 100 can utilize a variety of community detection models. For example, the signature discovery system 100 can utilize Leiden Community Detection, Louvain, Girvan-Newman, Infomap, or other community detection algorithms.

Moreover, FIG. 6 shows the signature discovery system 100 using MRMR feature selection 616 (minimum redundance maximum relevance). In one or more embodiments, the signature discovery system 100 uses MRMR feature selection 616 to generate a filtered community of similar genes from the community. In particular, MRMR feature selection 616 selects the most relevant features while ensuring minimal redundance among the selected features. For example, the signature discovery system 100 uses MRMR feature selection to identify the relevance of a feature (gene) and how well it correlates to sensitivity from applying the target compound. Further, the signature discovery system 100 identifies redundance of a feature (gene) by determining how much information it shares with other features (e.g., high redundance means the feature provides the same information as another feature, making it less valuable).

To illustrate, the signature discovery system 100 applies the MRMR feature selection 616 to define a target objective (e.g., gene expression sensitivity to a target compound), calculate a relevance score (shared information, correlation, information gain) for each gene in a community based on the target objective, and calculate the redundance of a gene (e.g., a high measure of shared information between the gene and another gene in the community). Moreover, the signature discovery system 100 can use MRMR feature selection 616 to rank the genes in a community based on their relevance to a target objective. In particular, the signature discovery system 100 can select the most relevant and the least redundant genes to generate a filtered community of genes. The signature discovery system 100 can utilize a variety of feature selection algorithms in addition to MRMR (e.g., Mutual Information or other models).

Accordingly, FIG. 6 shows the signature discovery system 100 transforming the data from the multi-modal ranked set of genes 602 using the overrepresentation analysis model 604, the community detection model 612, and the MRMR feature selection 616 to arrive at the gene signature 618. Thus, the signature discovery system 100 more effectively and accurately homes in on a subset of genes that correspond to gene expression sensitivity to a target compound.

In one or more embodiments, the signature discovery system 100 identified several gene signatures whose expression correlates with a target compound in Small Cell Lung Cancer (SCLC) cell lines and Patient-Derived Xenograft Organoid (PDXO) models. In particular, all identified signatures by the signature discovery system 100 strongly outperformed existing ten gene signatures previously used as a sensitivity biomarker for SCLC.

In some embodiments, identified signatures correlate with target compound sensitivity both across and within small cell lung cancer subtypes (e.g., small cell lung cancer can have differences in gene expression and epigenetic features and can be classified based on specific biomarkers or transcriptional signatures). In particular, small cell lung cancer subtypes can include NAPY subtypes which stands for NEUROD1 (e.g., transcription factor for neuronal differentiation such as neurogenesis and neuroendocrine differentiation), ASCL1 (e.g., transcription factor for differentiation of neuroendocrine cells), POU2F3 (e.g., transcription factor for differentiation of squamous cells), and YAP1 (e.g., transcriptional co-activator in the Hippo signaling pathway, which regulates cell proliferation, survival, and organ size).

For example, the NAPY subtypes play critical roles in the regulation of small cell lung cancer as their expression patterns are used to classify small cell lung cancer types. Thus, the gene signatures generated/identified by the signature discovery system 100 outperform simple molecular subtyping (e.g., simply categorizing biological samples by NAPY) as a target compound biomarker and may predict which SCLC-subtype (e.g., subtype A) tumors are more likely to be sensitive to the target compound.

In one or more embodiments, the signature discovery system 100 groups gene signatures by biological themes relevant to biology of the target compound. Moreover, the signature discovery system 100 generates gene signatures that are designed to be resilient to technical artifacts and small sample sizes, demonstrating the strongest performance in preclinical models that closely resemble patient samples e.g. better performance in PDXO vs. cell line datasets.

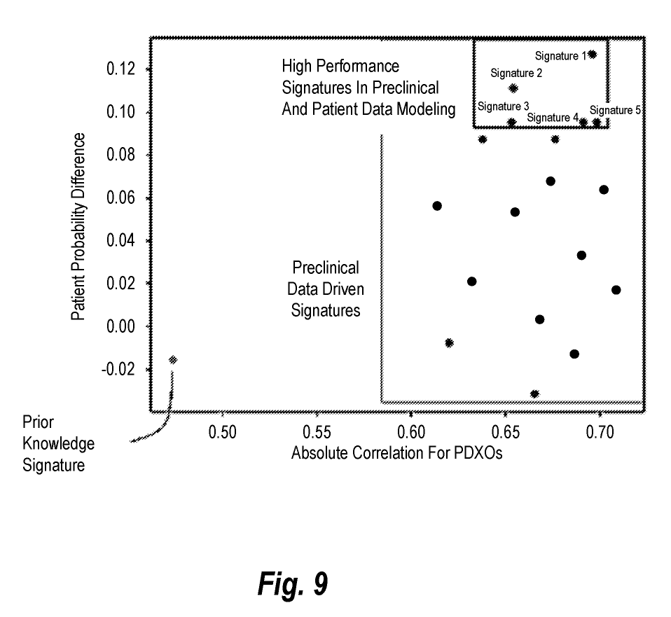

Furthermore, the signature discovery system 100 prioritizes gene signatures based on preclinical performance and causal modeling for the target compound (e.g., shown below in FIG. 9). For instance, scoring of genes by the signature discovery system 100 prioritized target compound signatures across two large, small cell lung cancer clinical datasets, and the signature discovery system 100 identified significant associations between patients with combined SCLC (cSCLC) histology or N0 stage (lacking lymph node metastasis) and less extreme target compound signature scores.

Moreover, in some embodiments, from modelling an inhibitor for the target compound signature scores as a proxy for responder status in the clinical patient SCLC dataset, experimenters found that excluding patients with combined small-cell lung cancer (cSCLC) histology or those without lymph node metastasis increased the responder rate from 32% to 39%. In one or more embodiments, the signature discovery system 100 identifies gene signatures that predict target compound inhibitor sensitivity within and/or across SCLC molecular subtypes. Moreover, as mentioned above, the signature discovery system 100 mines SCLC patient omic and clinical data for gene signatures (e.g., for a target compound for SCLC) to identify clinically actionable patient populations defined by tractable biomarkers (e.g., identifies patients with matching gene signatures, such that the target compound effectively targets disease in those patients).

In one or more embodiments, the signature discovery system 100 uses perturbation datasets that include a cell panel, a PDXO panel, and a PDXO with the target compound applied. In particular, the cell panel includes a plurality of SCLC cell lines treated with the target compound. Further, the PDXO panel includes a plurality of Patient-Derived Xenograft Organoids (PDXOs) treated with the target compound. Moreover, the PDXO with the target compound applied includes a plurality of organoid samples treated with target compound or treated with a vehicle (e.g., a control).

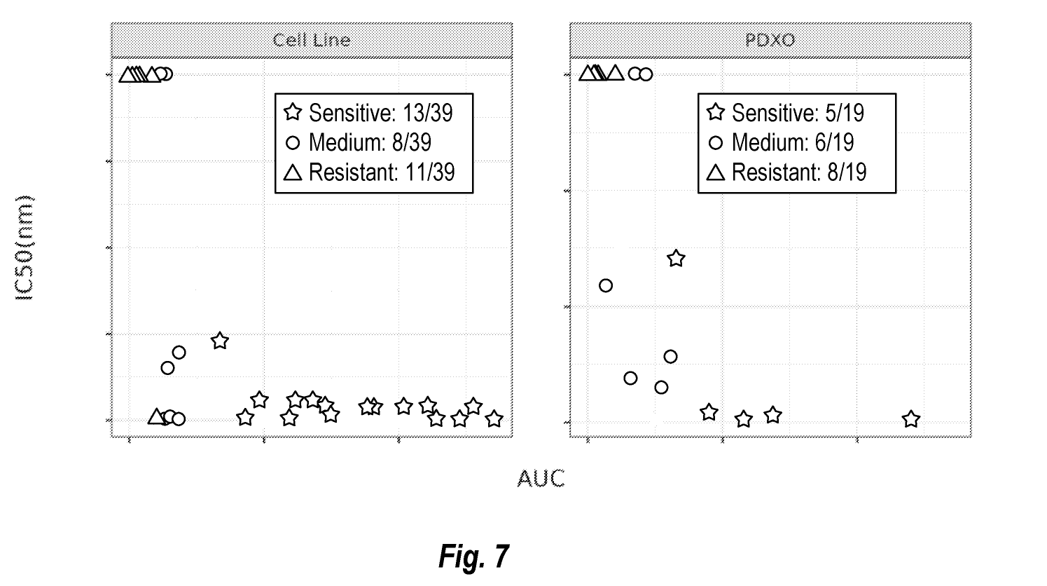

FIG. 7 shows sensitivity of cell line datasets compared with organoid datasets from applying a target compound in accordance with one or more embodiments. For example, the signature discovery system 100 identifies several gene signatures whose expression correlates with gene expression sensitivity to a target compound. In particular, the identified gene signatures correlate to small cell lung cancer cell line data and patient-derived xenograft organoid models (e.g., datasets).

FIG. 7 shows the relative sensitivity of a cell panel and a PDXO panel sample to a target compound inhibitor. In particular, FIG. 7 shows IC50 metrics versus the AUC metrics and shows how sensitive a potential sample is to the target compound inhibitor. For example, the chart on the left in FIG. 7 shows a maximal IC50 value (e.g., the data points at the top left of the left chart). Accordingly, samples of the cell line that have a maximal IC50 value are resistant to the target compound inhibitor. However, some of the cell line samples are in an intermediate state, where their IC50 values are still high, but they also have some signals showing high AUC metrics. In some embodiments, the medium sensitivities indicate an un-optimal assay setup. To distinguish these medium sensitivities, the signature discovery system 100 can perform the leave-one-out cross-validation to identify genes that are overly contributing.

Moreover, for the medium sensitivity samples, the signature discovery system 100 can further apply the bias weights to skew the signature discovery system 100 to the higher sensitivity samples. Thus, FIG. 7 illustrates that the IC50 values of the cell line dataset and the organoid dataset max out at different values as they used different maximal target compound concentrations. Thus, the signature discovery system 100 can rely on both data modalities to identify the most optimal subset of genes relative to a target compound based on applied sensitivity weights.

As mentioned above, the signature discovery system 100 generates the gene signatures and uses the gene signatures to identify clinical samples (e.g., patients) that match the identified subset of genes for the gene signatures (e.g., a target compound would effectively target a disease in a patient). In particular, the signature discovery system 100 uses multiple different clinical datasets.

In one or more embodiments, the signature discovery system 100 uses a first clinical dataset that includes RNA sequencing data, immunohistochemistry (IHC) for NEUROD1, ASCL1 and POU2F3 (e.g., the SCLC subtypes), and targeted exome sequencing. In particular, the majority of RNA sequencing samples in the first clinical dataset are biopsied from lymph nodes rather than lung tissue. Further, in some embodiments, the signature discovery system 100 uses a second clinical dataset that includes a plurality of patients with matched clinical metadata, targeted exome sequencing and RNA sequencing data, and all biopsies are primary tumors i.e. from the lung.

In one or more embodiments, the signature discovery system 100 generates gene signatures that strongly predicts sensitivity to the target compound across and within SCLC subtypes (e.g., NAPY subtypes) in preclinical models. Further, in some embodiments, the signature discovery system 100 generates gene signatures that most strongly perform in preclinical models that represent patient samples, i.e., patient-derived xenograft organoids, are likely better models of SCLC than cell lines. Moreover, the signature discovery system 100 generates gene signatures that are well expressed in patient samples (e.g., the first clinical data samples and the second clinical data samples), are robust to technical artifacts (e.g., batch effects, sample purity, etc.), robust to small sample sizes, and represents relevant biology (i.e., ideally a collection of related genes that relate to biology of the target compound).

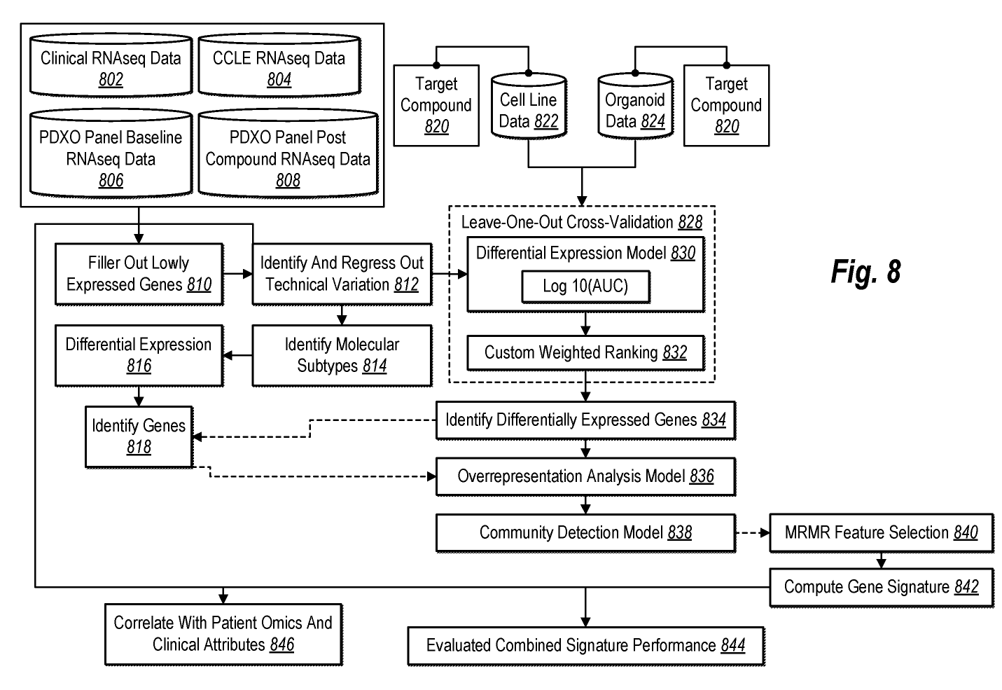

FIG. 8 illustrates a signature pipeline for filtering through and transforming various biological signals to compute a gene signature and correlate the gene signature with patient omics in accordance with one or more embodiments. For example, FIG. 8 shows the signature discovery system 100 accessing clinical data and pre-clinical datasets. In particular, FIG. 8 shows the signature discovery system 100 accessing clinical RNAseq data 802, Cancer Cell Line Encyclopedia (CCLE) RNAseq data 804, PDXO panel baseline RNAseq data 806 (e.g., measurements or data taken before treatment with the target compound), and PDXO panel post compound RNAseq data 808 (e.g., measurements taken after treatment with the target compound).

As shown in FIG. 8, the signature discovery system 100 performs an act 810 of filtering out the lowly expressed genes from the clinical data. For example, to ensure signatures are reliably expressed in SCLC patient samples, the signature discovery system 100 filtered out genes (e.g., using a differential expression model function that excludes genes with low or zero counts). To illustrate, the signature discovery system can filter out genes that do not have a minimum count of 10. In particular, the act 810 aids the signature discovery system 100 in generating robust expression measures and ignoring lowly expressed genes.

Moreover, the signature discovery system 100 performs an act 812 of identifying and regressing out technical variation. In particular, the act 812 aids the signature discovery system 100 in identifying genes that are robust to technical artifacts. For example, the signature discovery system 100 performs the act 812 by performing principal component analysis to determine which samples are clustering. Specifically, principal component analysis is a dimensionality reduction technique to simplify large datasets by transforming them into a smaller set of new variables to capture the most important variations in the data. In some embodiments, principal component analysis includes a first principal component (PC1) to capture the largest variance in the data, a second principal component (PC2) to capture the next largest variance (orthogonal/uncorrelated to the first component), and a third principal component (PC3) to capture the next largest variance (e.g., orthogonal to PC1 and PC2), and so on.

In one or more embodiments, the signature discovery system 100 performs principal component analysis and determines sample clustering without labels to identify why the samples are separate from each other for each stage of principal component analysis. In such cases, the signature discovery system 100 can identify potential other sources of variation within the data that are not tracked to known variables in the dataset (e.g., not tracked to how long the patient survived for or whether they are male or female, etc.). Moreover, in some cases, the signature discovery system 100 can utilize surrogate variable analysis (SVA) to identify surrogate variables within the datasets that could be contributing to technical variation. In particular, the signature discovery system 100 utilizes SVA to identify batch effects (e.g., elements in the datasets that need to be adjusted for when performing differential expression analysis).

Further, as shown, the signature discovery system 100 performs an act 814 of identifying molecular subtypes. In particular, the signature discovery system 100 identifies SCLC molecular subtypes based on nearest centroid (M3C) clustering. For instance, the signature discovery system 100 performs clustering and centroid-based techniques to group data points (e.g., data points from the clinical and pre-clinical datasets with filtered out genes that are lowly expressed) based on their similarity to the nearest centroid in a multidimensional space.

For example, the signature discovery system 100 selects an initial number of clusters (k), determines the average expression level for each gene across all samples to calculate the centroids, and then assigns each sample to a nearest centroid (e.g., computes the distance between the gene expression of a sample and each centroid). Moreover, the signature discovery system 100 can update centroids by recalculating mean expression values for each gene in each cluster. Thus, by using nearest centroid (M3C) clustering, the signature discovery system 100 performs the act 814 aids to generate strong predictions within and across molecular subtypes.

Moreover, as shown, the signature discovery system 100 performs an act 816 of determining differential expression for the target compound versus vehicle PDXOs (e.g., comparing test versus control) and further performs an act 818 of identifying genes. In particular, the act 818 includes identifying genes that are differentially expressed post application of the target compound and whose baseline expression correlates with target compound sensitivity.

As further shown on the right side of FIG. 8, the signature discovery system 100 can perform leave-one-out cross-validation 828 on cell line data 822 and organoid data 824 with the target compound 820 applied (as discussed above).

Moreover, the signature discovery system 100 uses a differential expression model 830 (e.g., with Log₁₀ AUC, with and without NAPY status) to generate gene differential expression measures. In one or more embodiments, the signature discovery system 100 utilizes a Limma-Voom differential expression pipeline that identifies genes up or down-regulated in sensitive vs. resistant SCLC models. Regression was performed using the log₁₀(AUC) measurements. In particular, the signature discovery system 100 utilizes AUC metrics as it incorporates both how sensitive samples are to a target compound, but also the potency of the target compound (e.g., IC50).

Moreover, in some embodiments, the signature discovery system 100 transforms the metrics via a log transformation to yield a more normally distributed sample. In some embodiments, the choice of Limma-Voom enabled the use of sample weights during model fitting. To bias differential expression results to those samples where we are most confident in relative sensitivity or resistance, the signature discovery system 100 inflated the weights with especially high AUC or low IC50 values.

In one or more embodiments, the signature discovery system 100 performs differential expression analysis by regressing the log₁₀(AUC) values, with and without NAPY subtype information as a covariate. This enabled the identification of genes that correlate with the target compound sensitivity within and across NAPY subtypes. As also shown in FIG. 8, in some embodiments, the signature discovery system 100 performs the act 812 at the step of using the differential expression model 830 for the cell line data 822 and the organoid data 824. In particular, the signature discovery system 100 performs principal component analysis to determine which samples are clustering in the cell line data 822 and the organoid data 824.

In one or more embodiments, experimenters determined stronger performance in PDXO versus cell line data samples. As highlighted above, in the cell line and PDXO datasets, many genes were commonly up- or downregulated in sensitive vs. insensitive samples. However, despite this agreement, at a relaxed false-discovery rate (FDR) of 10%, there was little agreement in the top differentially expressed genes. To combat this challenge, experimenters employed a custom ranking approach, where experimenters ranked differentially expressed genes in each dataset by their log fold change. In some embodiments, the signature discovery system 100 performs a custom weighted ranking 832 to identify differentially expressed genes common across datasets. In some embodiments, the custom weighted ranking 832 biases the rankings to the PDXO dataset.

As discussed above, these ranks were then averaged and compared to a null distribution of ranks generated via resampling. This enabled the identification of a single list (e.g., the multi-modal ranked list of genes) of differentially expressed genes across both datasets. Experimenters weighed the average of the ranks between the datasets (e.g., bias weights), enabling the lists of commonly differentially expressed genes across the datasets to be biased to the more clinically relevant PDXO samples.

As mentioned above and as shown in FIG. 8, the signature discovery system 100 identifies genes that are robust to technical artifacts. FIG. 8 shows the signature discovery system 100 performing an act 834 of identifying differentially expressed genes common across leave-one-out folds based on baseline RNAseq vs. target compound sensitivity (e.g., as indicated in FIG. 8, the signature discovery system 100 references the data from performing the act 818 while performing the act 834). In other words, the signature discovery system 100 identifies a subset of genes that are robust to removing individual samples from the datasets, as discussed above.

In particular, the signature discovery system 100 identifies outlier samples and known batch effects via PCA and RLE plots (relative log expression plots for normalization and assessing the quality of gene expression data). As mentioned above, in some embodiments, hidden technical variation was identified via surrogate variable analysis, as described in Leek, Johnson, Parker, Jaffe, Storey, The sva package for removing batch effects and other unwanted variation in high-throughput experiments, Bioinformatics, Volume 28, issue 6, Mar. 2012, pages 882-883. Overall, no significant surrogate variables were identified in the cell line and PDXO RNAseq datasets. In some embodiments, significant surrogate variables were identified in the cell line data 822 patient RNAseq dataset. This surrogate variable, along with tumor purity estimates, were regressed out of TPM matrices (e.g., transcripts per million matrix is used to represent gene expression levels and account for differences in sequencing depth and gene length allowing for more accurate comparisons of gene expression across different samples).

To aid interpretation and biological rationalization, the signature discovery system 100 groups the gene signatures by biological theme (e.g., samples sensitive to the target compound tend to possess high expression of a specific signature). To assemble gene signatures grouped by biological pathway, the signature discovery system 100 performs overrepresentation analysis on a list of differentially expressed genes (e.g., the multi-modal ranked set of genes).

As shown in FIG. 8, the signature discovery system 100 utilizes an overrepresentation analysis model 836 to generate sets of overrepresented genes. As overrepresented gene-sets can often overlap strongly in their component genes, the signature discovery system 100 clustered similar over-represented gene-sets into communities. As shown in FIG. 8, the signature discovery system 100 uses a community detection model 838 to generate one or more communities from the sets of overrepresented genes.

In particular, the signature discovery system 100 generates the one or more communities by calculating pairwise Jaccard similarity between the over-represented gene sets of all over-represented gene-sets. In some embodiments, the signature discovery system 100 uses a Jaccard value threshold of 0.2, enabling the construction of a network graph, where the vertices of similar gene-sets are connected to each other via edges with Jaccard similarity scores as weights. As mentioned above, the signature discovery system 100 uses a Leiden community detection on this network graph to identify communities of similar gene-sets.

Each community of gene-sets represents distinct biological themes. As discussed above, the signature discovery system 100 determines the union of over-represented genes in each community as a putative target compound signature and optionally performed additional MRMR feature selection. As shown in FIG. 8, the signature discovery system 100 uses MRMR feature selection 840 to filter out/remove one or more genes from one or more communities.

This optional step subsets the larger list of genes whose baseline expression levels correlates with target compound sensitivity for those genes whose expression is also target compound dependent. In doing so, this ensures that, in addition to baseline expression correlating with the target compound sensitivity, each signature gene is differentially expressed post target compound treatment in preclinical models. In some embodiments, the MRMR feature selection 840, was optionally employed to identify and remove genes from signatures that are contributing redundant information. In some embodiments, when performing the MRMR feature selection 840, the signature discovery system 100 weighted the MRMR selection towards features that were more relevant in PDXO samples.

In some embodiments, target compound-dependent genes were identified twice: once using all PDXO samples and again using only PDXO samples whose proliferation was found to be acutely sensitive to the target compound. Moreover, in some embodiments, repeating using only target compound sensitive PDXO samples ensures that the signature discovery system 100 enrich differentially expressed genes that might be driving target compound sensitivity. In particular, the signature discovery system 100 uses a rationale of MRMR feature selection 840 as this optional criterion prioritizes genes likely regulated by the target compound, either directly or indirectly, and thus more directly tied to target compound biology.

Further, from the community (e.g., filtered community) the signature discovery system 100 further computes a gene signature 842. As additionally shown in FIG. 8, the signature discovery system 100 can then perform an act 844 of evaluating a combined signature performance (e.g., assessing how well the subset of genes of the gene signature correlate with gene expression sensitivity to the target compound) or perform an act 846 of correlating the gene signature with patient omics and clinical attributes (e.g., identifying patients that have a matching subset of genes to the computed gene signature).

In one or more embodiments, the signature discovery system 100 correlates gene signatures to patient metadata. To identify clinical variables correlated to computed target compound signature scores, multiple 1-way ANOVA tests (analysis of variance, which is a statistical method to compare the means of three or more groups to determine if there is a significance difference between them) were computed between signature scores and selected clinical variables. To focus on the most impactful clinical variables, the signature discovery system 100 removes those with high redundancy, high missingness (>80%) and low variance (>95% a single value). As ANOVA tests require categorical independent variables, continuous variables were converted to low (minimum value -> Median - MAD), medium (Median - MAD -> Median + MAD) and high (Median + MAD -> maximal value) categories.

In other words, the signature discovery system 100 accesses clinical metadata, filters the metadata (e.g., based on high redundancy, high missingness, and low variance), converts continuous variables to categorical variables, determines pairwise interactions, filters metadata, and calculates signature scores between the filtered clinical metadata and the gene signatures, and generates the clinical RNAseq signature scores (e.g., a score indicating how well a gene signature performs relative to clinical samples, in other words a score associated with a gene signature indicates comparison of the signature across different data types (clinical data)).

SCLC preclinical models and patient samples can be divided into molecular subtypes based on the expression of four transcription factors: NEUROD1 (N), ASLC1 (A), POU2F3 (P) and YAP1 (Y), as described in Rudin, Poirier, Byers, et al., molecular subtypes of small cell lung cancer: a synthesis of human and mouse model data, Nature Review Cancer 19, 289-297, 2019. Moreover, in some embodiments, PDXO and cell lines datasets possessed comparable subtype compositions, with similar proportions to the dominant N, A and P subtypes. In particular, in some instances, the subtype A is most sensitive to a target compound in both the cell line dataset and the organoid dataset. Further, in some embodiments, the cell line dataset and the organoid dataset possess a broadly similar ratio of molecular subtypes.

In some embodiments, despite classifications being based on the expression of four genes (NAPY subtypes), NAPY subtypes neatly separated the whole transcriptome profiles of cell line and PDXO samples following principal component analysis (PCA). In other words, the expression patterns of the NAPY subtypes were able to clearly differentiate the gene expression profiles across the cell line data samples and the organoid data samples.

In one or more embodiments, the signature discovery system 100 performs differential expression analysis to compare target compound sensitivity versus target compound insensitive samples. In some embodiments, experimenters sought to identify genes whose baseline expression correlates with target compound sensitivity. In some embodiments, in comparing target compound sensitivity versus resistant samples, there was little overlap in differentially expressed genes (DEGs) between the cell line and PDXO datasets at a 10% FDR (false discovery rate). This was not entirely surprising, as experimenters expected the transcriptional profiles of cell lines and PDXOs to be very different. Despite these differences, changes of genes in the two datasets appeared modestly correlated. This correlation suggests, for example, that genes over-expressed in sensitive cell line samples also tended to be over-expressed in sensitive PDXO samples.

To obtain a common list of DEGs across both datasets, experimenters employed a custom ranking approach detailed above. To ensure this list of DEGs was not disproportionately dependent on the relative sensitivity or resistance of individual preclinical samples, whose sensitivity is likely subject to experimental variability and measurement error, experimenters repeated this procedure 58 times using leave-one-out sampling. As output, experimenters took forward a list of 108 consistently identified signature-generation genes that were as differentially expressed across leave-one-out folds.

In one or more embodiments, the signature discovery system 100 performs differential expression analysis of the target compound versus vehicle PDXOs. In some embodiments, the target compound regulates transcriptional responses predominantly via removing activating or inhibitory marks. To assess if genes whose baseline expression correlate with target compound sensitivity are also downstream targets of target compound transcriptional regulation, experimenters performed RNAseq on a plurality of PDXO samples treated with either the target compound or vehicle control. Despite differences in sequencing batches and computational pipelines, Principal Component Analysis (PCA) demonstrated impressive consistency in the PDXO transcriptional profiles, with existing baseline RNAseq profiles sitting close to Vehicle-treated PDXO samples.

In some embodiments, comparing the transcriptional profiles of the target compound and Vehicle samples, PCA identified modest shifts in PC1 and PC2. Consistent with this, differential expression analysis across all PDXO models identified many differentially expressed genes (DEGs) at an FDR of 5%. Over-representation analysis on the total list of DEGs across all samples identified strong enrichments for gene-sets and pathways reported to be regulated by the target compound. Interestingly, when analyzing each PDXO model separately, models with greater proliferative sensitivity to the target compound often had more DEGs. This trend is consistent with the most target compound-sensitive PDXO models being those where the target compound has the greatest activity.

In this comparison of differentially expressed genes (DEGs), experimenters found a small but statistically significant overlap between the genes altered in the target compound inhibitor treated patient-derived xenograft organoids (PDXOs) and those that showed significantly different baseline expression levels in target compound-sensitive versus insensitive PDXOs. Specifically, experimenters identified multiple overlapping genes, with a Fisher's exact p-value < 0.0001. Among these multiple genes, a subset displayed reciprocal expression changes across the two datasets. For instance, genes with higher baseline expression in target compound-sensitive PDXO models were downregulated following target compound treatment. These reciprocal expression changes are consistent with regulation by the target compound, prioritizing these subset of genes for downstream signature generation.

As shown in FIG. 9, the signature discovery system 100 generates a plurality of gene signatures. In particular, the signature discovery system 100 selects the top threshold (e.g., 5) signatures. As shown, compared to the “prior knowledge signature,” the gene signatures generated by the signature discovery system 100 have improved “absolute correlation for PDXOs” and “patient probability difference.” In other words, the gene signatures generated by the signature discovery system 100 significantly outperform prior knowledge signature in terms of absolute correlation for PDXOs and patient probability difference.

Following the methods outlined above, the experimenters identified two groups of signatures: (1) Signatures whose baseline expression levels correlate with the target compound sensitivity (2) Signature whose baseline expression levels correlate with target compound sensitivity AND whose expression significantly changes following target compound treatment. For each of the groups, signatures were using either a consensus list of differentially expressed genes or subsets of this consensus list of differentially expressed genes grouped by biological theme, with MRMR feature selection optionally applied to remove genes contributing redundant information.

To prioritize signatures, experimenters evaluated signature performance in preclinical and patient-level datasets. Specifically, experimenters selected signatures that: A) best correlated with target compound sensitivity in preclinical datasets, and B) using causal modeling, where signatures were differentially expressed in patients where lower target compound expression was determined to be causal in driving better survival. Using these two criteria, experimenters selected five top performing signatures for analysis.

Beyond these five signatures shown in the chart of FIG. 9, several signatures strongly correlated with the target compound sensitivity in preclinical datasets but just missed an FDR<0.1 differential expression cutoff in patient populations. In some instances, a signature can include genes whose expression not only correlated with target compound sensitivity in preclinical samples but whose expression was significantly changed in PDXO samples following target compound treatment. As suggested earlier, such target compound-dependent differential expression is consistent with direct or indirect regulation by the target compound and hints the signature is capturing relevant target compound biology. Thus, in some instances, along with the five already prioritized signatures shown in FIG. 9, this signature was also taken forward for further analysis (e.g., causal modeling).

Moreover, as shown in FIG. 10, in some embodiments, experimenters analyzed signature performance in preclinical datasets. As shown in FIG. 10, in both perturbation datasets, all prioritized signatures strongly outperformed an existing 10-gene signature reflecting cancer stemness, as measured by a stronger correlation between the signature and log₁₀AUC values. Moreover, signatures performed better in the more clinically relevant PDXO samples.

In particular, FIG. 10 shows the Pearson correlation of signature versus Log₁₀AUC for existing signatures and the signatures generated by the signature discovery system 100. For example, Pearson correlation is a statistical measure that quantifies the strength and direction of the linear relationship between two variables and is used to determine how strongly two variables are related to each other. Thus, FIG. 10 shows that the signatures generated by the signature discovery system 100 have a stronger correlation between gene signatures and AUC metrics than existing signatures.

In some embodiments, each of the prioritized signatures was associated with a biological theme and captured unique target compound-related biology. As an example, top-performing signature contains several genes known to downstream targets. Each of these signatures are strongly correlated with target compound sensitivity in cell and PDXO models. Critically, however, the prioritized signatures also correlated with target compound sensitivity within SCLC subtypes. Thus, these signatures provide greater granularity than SCLC subtypes in predicting target compound sensitivity. In other words, the gene signatures may be able to predict target compound sensitivity both across and within SCLC subtypes.

In one or more embodiments, the experimenters score target compound signatures in clinical datasets. Transcriptomic data will not be available prospectively for the target compound clinical trial. Therefore, experimenters aimed to identify feasible clinical biomarkers that could serve as substitutes for target compound signatures. To achieve this, experimenters explored whether the target compound signatures correlated with patient clinical attributes using two datasets that included matched clinical metadata, transcriptomic, and genomic profiles.

As mentioned above, the signature analysis identified six top-performing signatures that encompass various biological themes relevant to the target compound. As shown in FIG. 10, when the experimenters evaluated the target compound signatures across the two clinical datasets, the experimenters discovered that the expression of genes within these signatures correlated strongly with each other. Furthermore, in some embodiments, when experimenters compared the signature scores, experimenters observed correlations between the different signatures that closely resembled those found in cell line and patient-derived xenograft organoid (PDXO) datasets. These findings indicate that the signatures capture consistent transcriptomic patterns shared between preclinical and clinical datasets.

Beyond SCLC, experimenters also examined the expression of the prioritized target compound signature across a PanCancer cohort of >34000 RNAseq samples from >40 cancer types. Broadly, all target compound signatures exhibit some selectivity for SCLC patient samples. Moreover, in some embodiments, the signature discovery system 100 performs ANOVA analysis for clinical features versus signature scores. To identify clinical variables correlated to computed target compound signature scores, experimenters computed multiple 1-way ANOVA tests across signature scores vs selected individual or pairwise combinations (e.g. smoking + stage) of clinical features, as well as a small subset of molecular features (e.g. NAPY subtype). As expected, molecular features outperformed clinical features in both datasets, with all prioritized signatures strongly correlating with NAPY subtype.

By exclusively examining clinical features, experimenters identified three factors significantly associated with target compound signature scores. These features provide a patient stratification strategy to exclude those without those particular features.

Next, experimenters aimed to predict the impact of a straightforward patient stratification strategy on target compound inhibitor trial recruitment and responder rates. To estimate the responder status for the target compound, experimenters classified patients as responders if their target compound signature scores were above the mean signature score observed in sensitive cell lines and patient-derived xenograft (PDXO) models. By applying this criterion in the first clinical dataset of small cell lung cancer (SCLC) and excluding patients with cSCLC histology or N0 stage, experimenters increased the responder rate from 32% (in the overall cohort) to 39%.

When correlating signature scores with clinical metadata, experimenters make several simplifying assumptions that may not fully reflect biological realities. One key assumption is that signature scores derived from preclinical samples can directly translate to clinical samples. However, this may not hold true due to several factors. For instance, there are significant differences in the composition of bulk RNA sequencing samples; monoculture cells and patient-derived xenograft organoids differ substantially from the heterogeneous nature of actual patient samples, which include various components of the tumor microenvironment. Additionally, there are notable other in vivo versus in vitro differences, such as the distinction between growing cells on plastic compared to their natural environment in patient tissue. Batch effects during RNA sequencing processing can also introduce variability that affects the accuracy of our correlations.

To address these limitations, experimenters have employed several methods. For instance, experimenters utilized surrogate variable analysis (SVA) to eliminate additional sources of technical variation present in clinical datasets, such as batch effects. Additionally, experimenters used rank-based signature methods, which are more robust against these batch effects. Furthermore, experimenters regressed tumor purity estimates from patient RNA sequencing data in an effort to remove some of the variability caused by differing tumor microenvironment compositions.

In one or more embodiments, the signature discovery system 100 investigates the relationship between the target compound inhibitor gene signatures and genomic profiles, such as mutation status, using preclinical models like patient-derived xenografts (PDXOs) and cell line datasets, alongside patient samples. The rationale behind this investigation is that several target compound signatures have shown comparable performance in both preclinical models and causal modeling. By correlating these signatures with similar genomic aberrations across preclinical models and patient samples, experimenters can gain confidence in their validity. For instance, if signature A is found to correlate with MYC amplification, it would be reassuring to observe a similar correlation in cell lines and PDXO samples.

Additionally, in some embodiments, the signature discovery system 100 examines the expression of signature genes in tumor cells compared to stroma cells. Preclinical models, including PDXOs and cell lines, do not capture the tumor microenvironment, which consists of immune cells and cancer-associated fibroblasts (CAFs). In contrast, patient RNA sequencing samples do reflect this microenvironment. Ideally, the signature should show higher expression in tumor cells versus surrounding microenvironment cells, ensuring that the signature score calculated from clinical samples primarily represents tumor expression. This focus is important because preclinical models can only provide insights about genes whose expression in tumor cells correlates with target compound sensitivity. The signature discovery system 100 can achieve this goal by identifying suitable single-cell RNA sequencing datasets for SCLC and determining the genes that are differentially expressed between tumor and stroma cells. In one or more implementations, this information aids in regenerating target compound signatures, specifically targeting genes with high expression in SCLC tumor cells.

Furthermore, in some embodiments, the signature discovery system 100 computationally aligns preclinical and clinical transcriptomic datasets for better comparability. The signature discovery system 100 can address this challenge by using contrastive PCA to remove principal components associated with the tumor microenvironment, followed by mutual nearest neighbor batch correction to align preclinical and clinical datasets in the same transcriptomic space. In this manner, one or more implementations helps validate that signature scores derived from preclinical samples can be directly translated to clinical samples.

In some embodiments, the signature discovery system 100 identifies clinical features related to target compound signature scores could involve analyzing patient genomic attributes as well.

As mentioned above, the signature discovery system 100 also applies a target gene (e.g., target gene knockout) to one or more pre-clinical datasets to generate gene signatures. In one or more embodiments, the signature discovery system 100 uses a signature pipeline that identified gene signatures whose expression correlates with sensitivity to a gene knockout. In particular, the signature discovery system 100 uses a pipeline that involves a variety of processes to identify signatures that strongly predict CRISPR gene effects scores (through differential expression analysis), represent relevant biology (through over-representation analysis and community detection), are differentially expressed in tumor tissue (through differential expression analysis and removal of over-expressed genes in normal tissues), reflect elevated signatures in a significant percentage of patient tumor samples (by computing signatures across unaligned and aligned cell line, tumor, and normal tissue datasets), and have suitable cell lines available for validation that are representative of patient samples.

In one or more embodiments, the signature discovery system 100 at the early stage of trying to identify a target disease and biomarker hypotheses, suggests targets either internally or to an external partner (e.g., the target can be a target biomarker disease triple). In particular, the signature discovery system 100 can use target microsatellites for stable cancers (e.g., repetitive sequences) like colorectal cancers with a target compound, where the biomarker is microsatellite instability, and the indication is colorectal cancer.

In one or more embodiments, the signature discovery system 100 uses the signature pipeline to look beyond just simple genetic biomarkers (microsatellite instability or mutant status) and thus generate the gene expression signatures. As mentioned above, the gene signatures refer to the expression of a collection of genes that seem to be associated with a particular area of biology that predict how sensitive a cancer cell line would be to the loss of a potential gene (e.g., a gene target perturbation), and translating the genetic expression signature into a patient sample (e.g., a predictive gene expression signature).

In other words, the signature discovery system 100 uses the signature pipeline to identify gene signatures that correlate how sensitive a cell line might be to the loss of a particular gene target (e.g., the gene that is knocked out). In particular, the signature discovery system 100 generates gene signatures that strongly predict the Crispr gene effect scores. For example, the signature discovery system 100 generates gene signatures that change a lot between the very sensitive cell lines and the least sensitive cell lines.

In some embodiments, the signature discovery system 100 generates gene signatures for gene perturbations that are robust to any potential confounding factors that might be present in the experiment. In particular, the signature discovery system 100 generates a gene expression signature more likely to translate when taken outside of its original context (outside of the original cell lines) and also the gene expression signature performs better than a single gene.

In other words, the signature discovery system 100 generates/identifies a combination of the genes in the gene signature, where the expression of several different genes, is much better at predicting how sensitive the genes are to a target perturbation than any individual gene. Moreover, the gene signature represents relevant biology as the subset of genes are not just a collection of random genes that predict how well a cell line is sensitive to the Crispr of target X, but rather they might be clustered together by a particular biological theme or pathway.

In one or more embodiments, the signature discovery system 100 screens RNA data (e.g., screens all genes in the genome and looking at whether cell lines with a target perturbation applied have genes that die or do not die in response). In particular, the signature discovery system 100 performs differential expression analysis (e.g., to analyze which genes are higher or lower in the sensitive samples for a particular gene versus the insensitive samples). To illustrate, the signature discovery system 100 uses CRISPR gene effect scores (e.g., scores that quantify the impact of gene knockouts), and expression data, performs differential expression analysis by CRISPR gene effect per indication, takes the list a list of differentially expressed genes, adjusts for potential confounding factors, and then performs overrepresentation analysis and community detection. For example, the signature discovery system 100 obtains a list of filtered genes from the community detection (e.g., and MRMR feature selection) to compute the gene signatures (e.g., to compute a unified signature that is up or down regulated to the sensitive or insensitive cell lines of the target of interest).

In one or more embodiments, the signature discovery system 100 ensures that the biomarker is elevated in patient samples compared to the non-patient samples. In particular, the signature discovery system 100 confirms that there is a threshold percentage of patients that would be positive for the biomarker. For example, the signature discovery system 100 pulls in publicly available RNA sequencing data and after performing some alignment of the data using a set aligner (to aid in correcting batch effects and also technical differences between the experiments), the signature discovery system 100 performs differential expression analysis on tumor versus normal to get a list of genes that are overexpressed or under expressed in patient tumor samples. In other words, the signature discovery system 100 compares the differential expression measures with patient clinical data (e.g., cancer cell lines) to generate/compute the gene signatures across unaligned and aligned cell lines, tumor, and normal tissue datasets.

In one or more embodiments, the signature discovery system 100 identifies biomarkers that prioritize genes that are not only just predictive of how sensitive a cell line is to the potential Crispr or potential of a target, but also that those genes are overexpressed in patient samples. In other words, the biomarker is likely to be useful for stratifying tumor-based patients, as to whether they would be high or low for the for the biomarker.

Accordingly, the signature discovery system 100 generates/computes signatures across unaligned and aligned cell line, tumor, and normal tissue datasets by ranking a subset of genes identified for a signature (based on the process discussed above) and computing a score for the ranked subset of genes. In some embodiments, the signature discovery system 100 generates a signature score for the subset of genes relative to all genes from which the subset of genes originated (e.g., the original dataset). In particular, signature score can range from 0.5 to -0.5 where zero = evenly distributed, 0.5 = the list of genes is more enriched for the higher expressed genes, -0.5 = the list of genes is more enriched for the lower expressed genes.

Thus, the signature discovery system 100 can generate an individual signature score which can correlate with a particular patient or a combined signature score which can indicate an estimate of whether a patient is sensitive to a target perturbation (e.g., calculated from a linear regression between the individual signature score and the combined signature score).

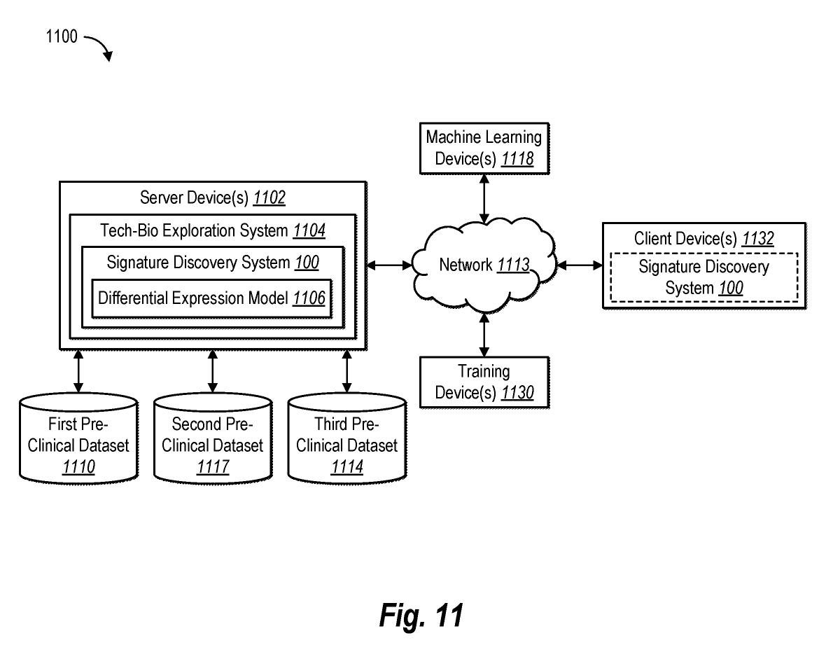

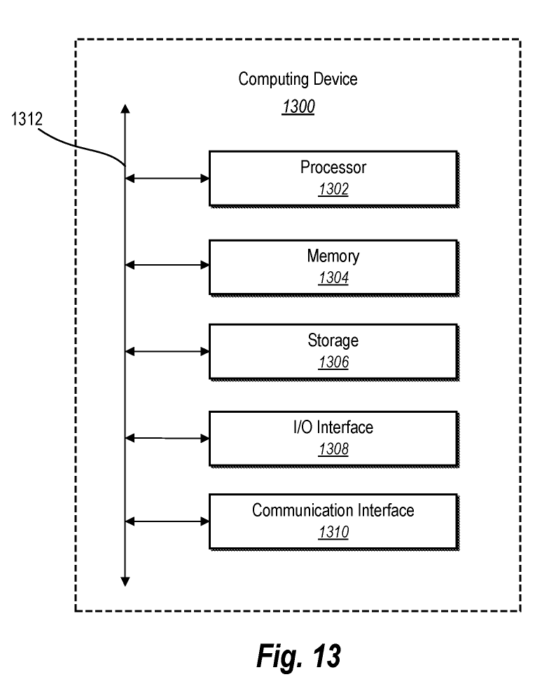

As shown in FIG. 11, an environment 1100 includes server device(s) 1102 (which includes a tech-bio exploration system 1104 and the signature discovery system 100), a network 1113, and client device(s) 1132. As further illustrated in FIG. 11, the various computing devices within the environment 1100 can communicate via the network 1113. Although FIG. 11 illustrates the signature discovery system 100 being implemented by a particular component and/or device within the environment, the signature discovery system 100 can be implemented, in whole or in part, by other computing devices and/or components in the environment 1100 (e.g., the additional device(s)). Additional description regarding the illustrated computing devices is provided with respect to FIG. 13 below.

As shown in FIG. 11, the server device(s) 1102 (e.g., one or more local servers operated by a particular entity) can include the tech-bio exploration system 1104. In some embodiments, the tech-bio exploration system 1104 can determine, store, generate, and/or display tech-bio information including target compounds, pre-clinical datasets, clinical datasets, gene differential expression measures, initial ranked sets of genes, multi-modal ranked sets of genes, overrepresented gene sets, communities of genes, gene signatures, patient sample data compared with gene signatures, drug discovery processes, maps of biology, experiments from various sources, and/or machine learning tech-bio predictions. For instance, the tech-bio exploration system 1104 can analyze data with a target compound applied, gene signatures (e.g., compared with patient data samples), data signals corresponding to various treatments or interventions (e.g., compounds or biologics) and the corresponding relationships in genetics, proteomics, phenomics (i.e., cellular phenotypes), and invivomics (e.g., expressions or results within a living animal). Moreover, the tech-bio exploration system 1104 provides an environment for operating, executing, and managing complex drug discovery pipelines (e.g., hit-to-lead and lead optimization).

For instance, the tech-bio exploration system 1104 can generate and access experimental results corresponding to gene sequences, protein shapes/folding, protein/compound interactions, phenotypes resulting from various interventions or perturbations (e.g., gene knockout sequences or compound treatments), and/or invivo experimentation on various treatments in living animals. By analyzing these signals (e.g., utilizing various machine learning models), the tech-bio exploration system 1104 can generate or determine a variety of predictions and inter-relationships for improving treatments/interventions (e.g., generating a set of target molecular structures for initiating compound drug discovery processes).

To illustrate, the tech-bio exploration system 1104 can generate gene signatures (e.g., to identify relevant patients for target compound treatment), maps of biology indicating biological inter-relationships, or similarities between these various input signals to discover potential new treatments as part of the complex compound discovery process. For example, the tech-bio exploration system 1104 can utilize machine learning and/or maps of biology to identify a target compound, generate gene differential expression measures, and further generate one or more gene signatures. The tech-bio exploration system 1104 can then identify new programs/treatments to initiate based on gene signatures or can identify patient populations to apply a target compound based on the gene signature.

The tech-bio exploration system 1104 can generate GUIs comprising dynamic user interface elements to convey tech-bio information and receive user input for intelligently exploring tech-bio information. Indeed, as mentioned above, the tech-bio exploration system 1104 can generate GUIs displaying different maps of biology that intuitively and efficiently express complex interactions between different biological systems for identifying improved treatment solutions. Furthermore, the tech-bio exploration system 1104 can also electronically communicate tech-bio information between various computing devices.

As shown in FIG. 11, the tech-bio exploration system 1104 can include a system that facilitates various models or algorithms for analyzing data transformations (e.g., gene responses to target compound). In particular, FIG. 11 shows the signature discovery system 100 that includes a differential expression model 1106 (e.g., Log₁₀AUC). Furthermore, the tech-bio exploration system 1104 can generate maps of biology (e.g., maps or visualizations illustrating similarities or relationships between genes, proteins, diseases, compounds, and/or treatments) and discovering new treatment options over one or more networks. For example, the tech-bio exploration system 1104 collects, manages, and transmits data across a variety of different entities, accounts, and devices. In some cases, the tech-bio exploration system 1104 is a network system that facilitates access to (and analysis of) tech-bio information within a centralized operating system. Indeed, the tech-bio exploration system 1104 can link data from different network-based research institutions to generate and analyze gene differential expression measures and also generate gene signatures.

As shown in FIG. 11, the tech-bio exploration system 1104 can include a system that comprises the signature discovery system 100 that generates, stores, manages, transmits data pertaining to applying a target compound (or target perturbation) to a first pre-clinical dataset 1110, a second pre-clinical dataset 1117 and a third pre-clinical dataset 1114 (e.g., that relate to different data modalities). For example, in context of the above description for the tech-bio exploration system 1104, in some embodiments the tech-bio exploration system 1104 further utilizes the signature discovery system 100 to enhance the coordination between various groups involved in the drug discovery process. For instance, the signature discovery system 100 works in tandem with the tech-bio exploration system 1104 to generate gene signatures that indicate a subset of genes correlating to gene expression sensitivity to a target compound.

To illustrate, the signature discovery system 100 utilizes the differential expression model 1106 to analyze AUC metrics and/or IC50 metrics, perform data transformations on the data, model the data, and generate gene differential expression measures. Specifically, the signature discovery system 100 ranks the genes according to the gene differential expression measures (e.g., using sensitivity weights or bias weights) to generate a multi-modal ranked set of genes. As mentioned above, the signature discovery system 100 generates the multi-modal ranked set of genes and then generates a gene signature from the ranked list. To further illustrate, the tech-bio exploration system 1104 utilizes the signature discovery system 100 at the program discovery phase to identify additional potential compounds based on the generated gene signature (e.g., overlapping biological pathways that could target another drug). Moreover, in some embodiments, the tech-bio exploration system 1104 utilizes the signature discovery system 100 at the hit-to-lead phase (e.g., where a set of feasible compounds have already been identified based on the gene signature) and performs additional iterations of the feasible compounds to refine the set of feasible compounds (e.g., narrow down the list by exploring the state space and prioritizing exploration or prioritizing greedier actions, e.g., higher reward actions).

As also illustrated in FIG. 11, the environment includes the client device(s) 1132. As mentioned above, the client device(s) 1132 can be involved in the process of drug discovery. Thus, for example, the client device(s) 1132 can coordinate/manage generating a particular target compound or target perturbation for further downstream testing (e.g., such as generating a gene signature for the target compound). For instance, the client device(s) 1132 can coordinate/ manage testing a target compound under various conditions to further determine whether to initiate one or more programs (industrial program generation or industrial compound generation).

To illustrate, the client device(s) 1132 can include computing devices that implement or manage a compound program generation stage of a compound discovery process. Similarly, the client device(s) 1132 can include computing devices that implement or manage a compound lead generation stage and the client device(s) 1132 can include computing devices that implement or manage a compound/dose selection stage. For example, the signature discovery system 100 can receive one or more requests to generate a gene signature according to a selected target compound.

In some embodiments, the environment also includes additional device(s). For example, the signature discovery system 100 can utilize the additional device(s) to further operate and manage downstream operations after generating gene signatures. For instance, the additional device(s) include experimental device(s) and analytical device(s). Further, in some instances, the additional device(s) also include the computing devices discussed below in FIG. 13.

Furthermore, in one or more implementations, the client device(s) 1132 include a client application. The client application can include instructions that (upon execution) cause the client device(s) 1132 to perform various actions. For example, a user of a user account can interact with the client application on the client device(s) 1132 to execute the generation of a gene signature. For instance, in some embodiments the signature discovery system 100 receives a request to generate a gene signature. In response, the signature discovery system 100 analyzes gene differential expression measures for data with a target compound applied and further generates the gene signature and returns the gene signature to the client device(s) 1132 along with additional options to compare the gene signature with patient cancer samples. In some instances, the transmittal of the gene signature (along with corresponding analytical data) to the client device(s) 1132 causes the client device(s) 1132 to further present options for executing an action (e.g., performing downstream experiments, tests, or evaluations of the gene signature).

In one or more embodiments, the environment 1100 can also include dedicated training device(s) 1130. For example, the dedicated training device(s) 1130 can include computing devices or virtual machines dedicated to training or implementing the differential expression model 1106 and/or machine learning models used to generate gene signature. For example, the dedicated training device(s) 1130 can provide datasets, parameters, objectives, and other learning constraints to train the machine learning models to generate outputs specific to a task. Thus, the signature discovery system 100 interacts with the dedicated training device(s) 1130 to perform a variety of tasks related to generating gene signatures.

As shown in FIG. 11, the environment 1100 includes machine learning device(s) 1118. In particular, the machine learning device(s) 1118 are attuned for specific objectives (e.g., downstream analysis), and the signature discovery system 100 accesses the training device(s) 1130 to train/fine-tune the machine learning device(s) 1118. In some embodiments, the signature discovery system 100 utilizes the machine learning device(s) 1118 to generate a target compound related to a specific disease.

The environment can also include experimental device(s). For example, the tech-bio exploration system 1104 can interact with the experimental device(s) that include intelligent robotic devices and camera devices for applying a target compound to a plurality of data samples, generating and capturing digital images of cellular phenotypes resulting from different perturbations (e.g., genetic knockouts or compound treatments of stem cells). Similarly, the experimental device(s) can include camera devices and/or other sensors (e.g., heat or motion sensors) capturing real-time information from animals as part of invivo experimentation. The tech-bio exploration system 1104 can also interact with a variety of other experimental device(s) such as devices for determining, generating, or extracting gene sequences or protein information. For example, the experimental device(s) may include computing devices linked to biosensorselectrophysiological platforms, x-ray crystallography machines, liquid chromatography mass spectrometry systems, nuclear magnetic resonance spectrometers, mass spectrometers. In some implementations, the signature discovery system 100 generates tractability scores and further determines to employ or utilize one or more experimental devices (e.g., to initiate one or more experiments based on the tractability scores).

As further shown in FIG. 11, the environment includes the network 1113. As mentioned above, the network 1113 can enable communication between components of the environment. In one or more embodiments, the network 1113 may include a suitable network and may communicate using a various number of communication platforms and technologies suitable for transmitting data and/or communication signals, examples of which are described with reference to FIG. 11. Furthermore, although FIG. 11 illustrates computing devices communicating via the network 1113, the various components of the environment can communicate and/or interact via other methods (e.g., communicate directly).

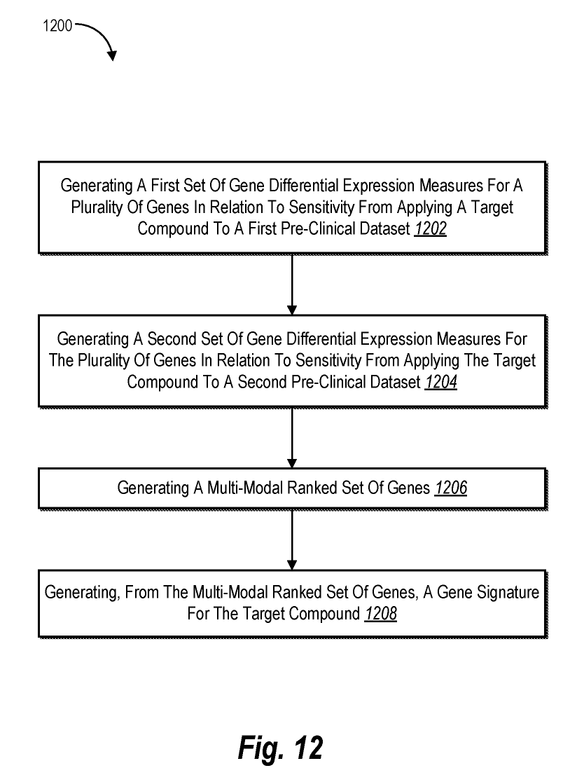

FIGS. 1-11, the corresponding text, and the examples provide a number of different systems, methods, and non-transitory computer readable media for utilizing signature discovery system 100 to generate a gene signature. In addition to the foregoing, embodiments can also be described in terms of flowcharts comprising acts for accomplishing a particular result. For example, FIG. 12 illustrates a flowchart of an example sequence of acts in accordance with one or more embodiments.

While FIG. 12 illustrates acts according to some embodiments, alternative embodiments may omit, add to, reorder, and/or modify any of the acts shown in FIG. 12. The acts of FIG. 12 can be performed as part of a method (e.g., a computer-implemented method). Alternatively, a non-transitory computer readable medium can comprise instructions, that when executed by one or more processors (e.g., at least one processor), cause a computing device to perform the acts of FIG. 12. In still further embodiments, a system can perform the acts of FIG. 12. Additionally, the acts described herein may be repeated or performed in parallel with one another or in parallel with different instances of the same or other similar acts.

FIG. 12 illustrates an example series of acts 1200 for generating a gene signature in accordance with one or more embodiments. The series of acts 1200 can include an act 1202 of generating a first set of gene differential expression measures for a plurality of genes in relation to sensitivity from applying a target compound to a first pre-clinical dataset, an act 1204 of generating a second set of gene differential expression measures for the plurality of genes in relation to sensitivity from applying the target compound to a second pre-clinical dataset, an act 1206 of generating a multi-modal ranked set of genes, and an act 1208 of generating, from the multi-modal ranked set of genes, a gene signature for the target compound.

Specifically, the series of acts 1200 can include acts of generating, utilizing a differential expression model, a first set of gene differential expression measures for a plurality of genes in relation to sensitivity from applying a target compound in a first pre-clinical dataset corresponding to a first data modality; generating, utilizing the differential expression model, a second set of gene differential expression measures for the plurality of genes in relation to sensitivity from applying the target compound to a second pre-clinical dataset corresponding to a second data modality; generating a multi-modal ranked set of genes from the first set of gene differential expression measures and the second set of gene differential expression measures; and generating, from the multi-modal ranked set of genes utilizing an overrepresentation analysis model, a gene signature for the target compound, wherein the gene signature comprises a subset of genes from the multi-modal ranked set of genes that correlate with gene expression sensitivity to the target compound.

In one or more embodiments, the series of acts 1200 includes generating area under the curve metrics from dose-response curves in applying the target compound in the first pre-clinical dataset; and generating the first set of gene differential expression measures from the area under the curve metrics. In one or more implementations, the series of acts 1200 includes determining area under the curve metrics or inhibitory concentration metrics for samples from the first pre-clinical dataset and the second pre-clinical dataset; and generating the first set of gene differential expression measures by applying sensitivity weights to a subset of samples from the first pre-clinical dataset and the second pre-clinical dataset based on the area under the curve metrics or the inhibitory concentration metrics.

In addition, in one or more implementations, the series of acts 1200 includes generating the first set of gene differential expression measures comprises generating, utilizing the differential expression model, the first set of gene differential expression measures from a cell line data set comprising expression data from applying the target compound to a plurality of cells from a plurality of cell lines; and generating the second set of gene differential expression measures comprises generating, utilizing the differential expression model, the second set of gene differential expression measures from an organoid data set comprising expression data from applying the target compound to a plurality of xenograft organoids.

Further, in some implementations, the series of acts 1200 includes generating a first ranked list of genes and a second ranked list of genes by ranking the first set of gene differential expression measures and the second set of gene differential expression measures; and combining the first ranked list of genes and the second ranked list of genes to generate the multi-modal ranked set of genes.

In one or more implementations, the series of acts 1200 includes applying a bias weight to the first ranked list of genes or to the second ranked list of genes. Moreover, in one or more implementations, the series of acts 1200 includes generating an initial multi-modal ranked set of genes from the first ranked list of genes and the second ranked list of genes; determining statistical significance measures for the initial multi-modal ranked set of genes by comparing the initial multi-modal ranked set of genes to a null distribution of ranks; and filtering one or more genes from the initial multi-modal ranked set of genes utilizing the statistical significance measures to generate the multi-modal ranked set of genes.

In addition, in some implementations, the series of acts 1200 includes filtering one or more genes utilizing leave-one-out cross-validation by: iteratively removing individual samples from the first pre-clinical dataset or the second pre-clinical dataset; and comparing resulting gene differential expression measures for the first pre-clinical dataset and the second pre-clinical dataset with the individual samples removed. In one or more implementations, the series of acts 1200 includes comparing, utilizing the overrepresentation analysis model, the multi-modal ranked set of genes with a population of genes corresponding to biological pathways to identify overrepresented gene sets from the multi-modal ranked set of genes for the biological pathways.

Moreover, in one or more implementations, the series of acts 1200 includes generating similarity scores between the overrepresented gene sets for the biological pathways; generating a graph for the overrepresented gene sets, wherein the graph comprises vertices indicating the overrepresented gene sets for the biological pathways and edges indicating the similarity scores; and generating, utilizing a community detection model, a community of similar gene sets from the graph for the overrepresented gene sets.

Moreover, in one or more implementations, the series of acts 1200 includes generating a filtered community of similar genes by filtering, utilizing a minimum redundance maximum relevance feature selection, one or more genes from the community of similar genes; and generating the gene signature for the target compound from the filtered community of similar genes.

In one or more implementations, the series of acts 1200 includes generating, utilizing a differential abundance model, a first set of differential abundance measures for a plurality of biomolecules in relation to sensitivity from applying a target compound in a first pre-clinical dataset corresponding to a first data modality; generating, utilizing the differential abundance model, a second set of differential abundance measures for the plurality of biomolecules in relation to sensitivity from applying the target compound to a second pre-clinical dataset corresponding to a second data modality; generating a multi-modal ranked set of biomolecules from the first set of differential abundance measures and the second set of differential abundance measures; and generating, from the multi-modal ranked set of biomolecules utilizing an overrepresentation analysis model, a biomolecule signature for the target compound, wherein the biomolecule signature comprises a subset of biomolecules from the multi-modal ranked set of biomolecules that correlate with biomolecule abundance sensitivity to the target compound.

In one or more implementations, the series of acts 1200 includes determining area under the curve metrics or inhibitory concentration metrics for samples from the first pre-clinical dataset and the second pre-clinical dataset; and generating the first set of differential abundance measures by applying sensitivity weights to a subset of samples from the first pre-clinical dataset and the second pre-clinical dataset based on the area under the curve metrics or the inhibitory concentration metrics.

In one or more implementations, the series of acts 1200 includes generating a first ranked list of biomolecules and a second ranked list of biomolecules by ranking the first set of differential abundance measures and the second set of differential abundance measures; and combining the first ranked list of biomolecules and the second ranked list of biomolecules to generate the multi-modal ranked set of biomolecules, wherein combining the first ranked list of biomolecules and the second ranked list of biomolecules to generate the multi-modal ranked set of biomolecules comprises applying a bias weight to the first ranked list of biomolecules relative or to the second ranked list of biomolecules.

In one or more implementations, the series of acts 1200 includes generating an initial multi-modal ranked set of biomolecules from the first ranked list of biomolecules and the second ranked list of biomolecules; determining statistical significance measures for the initial multi-modal ranked set of biomolecules by comparing the initial multi-modal ranked set of biomolecules to a null distribution of ranks; and filtering one or more biomolecules from the initial multi-modal ranked set of biomolecules utilizing the statistical significance measures to generate the multi-modal ranked set of biomolecules.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 13 illustrates a block diagram of an example computing device 1300 that may be configured to perform one or more of the processes described above. One will appreciate that one or more computing devices, such as the computing device 1300 may represent the computing devices described above. In one or more embodiments, the computing device 1300 may be a mobile device (e.g., a mobile telephone, a smartphone, a PDA, a tablet, a laptop, a camera, a tracker, a watch, a wearable device, etc.). In some embodiments, the computing device 1300 may be a non-mobile device (e.g., a desktop computer or another type of client device). Further, the computing device 1300 may be a server device that includes cloud-based processing and storage capabilities.

As shown in FIG. 13, the computing device 1300 can include one or more processor(s) 1302, memory 1304, a storage device 1306, input/output interfaces 1308 (or “I/O interfaces 1308”), and a communication interface 1310, which may be communicatively coupled by way of a communication infrastructure (e.g., bus 1312). While the computing device 1300 is shown in FIG. 13, the components illustrated in FIG. 13 are not intended to be limiting. Additional or alternative components may be used in other embodiments. Furthermore, in certain embodiments, the computing device 1300 includes fewer components than those shown in FIG. 13. Components of the computing device 1300 shown in FIG. 13 will now be described in additional detail.

In particular embodiments, the processor(s) 1302 includes hardware for executing instructions, such as those making up a computer program. As an example, and not by way of limitation, to execute instructions, the processor(s) 1302 may retrieve (or fetch) the instructions from an internal register, an internal cache, memory 1304, or a storage device 1306 and decode and execute them.

The computing device 1300 includes memory 1304, which is coupled to the processor(s) 1302. The memory 1304 may be used for storing data, metadata, and programs for execution by the processor(s). The memory 1304 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1304 may be internal or distributed memory.

The computing device 1300 includes a storage device 1306 includes storage for storing data or instructions. As an example, and not by way of limitation, the storage device 1306 can include a non-transitory storage medium described above. The storage device 1306 may include a hard disk drive (HDD), flash memory, a Universal Serial Bus (USB) drive or a combination these or other storage devices.

As shown, the computing device 1300 includes one or more I/O interfaces 1308, which are provided to allow a user to provide input to (such as user strokes), receive output from, and otherwise transfer data to and from the computing device 1300. These I/O interfaces 1308 may include a mouse, keypad or a keyboard, a touch screen, camera, optical scanner, network interface, modem, other known I/O devices or a combination of such I/O interfaces 1308. The touch screen may be activated with a stylus or a finger.

The I/O interfaces 1308 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1308 are configured to provide graphical data to a display for presentation to a user. The graphical data may be representative of one or more graphical user interfaces and/or any other graphical content as may serve a particular implementation.

The computing device 1300 can further include a communication interface 1310. The communication interface 1310 can include hardware, software, or both. The communication interface 1310 provides one or more interfaces for communication (such as, for example, packet-based communication) between the computing device and one or more other computing devices or one or more networks. As an example, and not by way of limitation, communication interface 1310 may include a network interface controller (NIC) or network adapter for communicating with an Ethernet or other wire-based network or a wireless NIC (WNIC) or wireless adapter for communicating with a wireless network, such as a WI-FI. The computing device 1300 can further include a bus 1312. The bus 1312 can include hardware, software, or both that connects components of computing device 1300 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 1300 can include a client device that includes a requester application or a web browser, such as MICROSOFT INTERNET EXPLORER, GOOGLE CHROME, or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 1104 may include a variety of servers, sub-systems, programs, modules, logs, and data stores. In particular embodiments, the tech-bio exploration system 1104 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 1104 may also include suitable components such as network interfaces, security mechanisms, load balancers, failover servers, management-and-network-operations consoles, other suitable components, or any suitable combination thereof. In particular embodiments, the tech-bio exploration system 1104 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 1104 and one or more client devices. An action logger may be used to receive communications from a web server about a user’s actions on or off the tech-bio exploration system 1104. In conjunction with the action log, a third-party-content-object log may be maintained of user exposures to third-party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 1104. A privacy setting of a user determines how particular information associated with a user can be shared. The authorization server may allow users to opt in to or opt out of having their actions logged by the tech-bio exploration system 1104 or shared with other systems, such as, for example, by setting appropriate privacy settings. Third-party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating, utilizing a differential expression model, a first set of gene differential expression measures for a plurality of genes in relation to sensitivity from applying a target compound in a first pre-clinical dataset corresponding to a first data modality;
generating, utilizing the differential expression model, a second set of gene differential expression measures for the plurality of genes in relation to sensitivity from applying the target compound to a second pre-clinical dataset corresponding to a second data modality;
generating a multi-modal ranked set of genes from the first set of gene differential expression measures and the second set of gene differential expression measures; and
generating, from the multi-modal ranked set of genes utilizing an overrepresentation analysis model, a gene signature for the target compound, wherein the gene signature comprises a subset of genes from the multi-modal ranked set of genes that correlate with gene expression sensitivity to the target compound.

2. The computer-implemented method of claim 1, wherein generating the first set of gene differential expression measures comprises:
generating area under the curve metrics from dose-response curves in applying the target compound in the first pre-clinical dataset; and
generating the first set of gene differential expression measures from the area under the curve metrics.

3. The computer-implemented method of claim 1, further comprising:
determining area under the curve metrics or inhibitory concentration metrics for samples from the first pre-clinical dataset and the second pre-clinical dataset; and
generating the first set of gene differential expression measures by applying sensitivity weights to a subset of samples from the first pre-clinical dataset and the second pre-clinical dataset based on the area under the curve metrics or the inhibitory concentration metrics.

4. The computer-implemented method of claim 1, wherein:
generating the first set of gene differential expression measures comprises generating, utilizing the differential expression model, the first set of gene differential expression measures from a cell line data set comprising expression data from applying the target compound to a plurality of cells from a plurality of cell lines; and
generating the second set of gene differential expression measures comprises generating, utilizing the differential expression model, the second set of gene differential expression measures from an organoid data set comprising expression data from applying the target compound to a plurality of xenograft organoids.

5. The computer-implemented method of claim 1, wherein generating the multi-modal ranked set of genes comprises:
generating a first ranked list of genes and a second ranked list of genes by ranking the first set of gene differential expression measures and the second set of gene differential expression measures; and
combining the first ranked list of genes and the second ranked list of genes to generate the multi-modal ranked set of genes.

6. The computer-implemented method of claim 5, wherein combining the first ranked list of genes and the second ranked list of genes to generate the multi-modal ranked set of genes comprises applying a bias weight to the first ranked list of genes or to the second ranked list of genes.

7. The computer-implemented method of claim 5, wherein generating the multi-modal ranked set of genes comprises:
generating an initial multi-modal ranked set of genes from the first ranked list of genes and the second ranked list of genes;
determining statistical significance measures for the initial multi-modal ranked set of genes by comparing the initial multi-modal ranked set of genes to a null distribution of ranks; and
filtering one or more genes from the initial multi-modal ranked set of genes utilizing the statistical significance measures to generate the multi-modal ranked set of genes.

8. The computer-implemented method of claim 5, wherein generating the first ranked list of genes and the second ranked list of genes comprises filtering one or more genes utilizing leave-one-out cross-validation by:
iteratively removing individual samples from the first pre-clinical dataset or the second pre-clinical dataset; and
comparing resulting gene differential expression measures for the first pre-clinical dataset and the second pre-clinical dataset with the individual samples removed.

9. The computer-implemented method of claim 1, wherein generating the gene signature comprises comparing, utilizing the overrepresentation analysis model, the multi-modal ranked set of genes with a population of genes corresponding to biological pathways to identify overrepresented gene sets from the multi-modal ranked set of genes for the biological pathways.

10. The computer-implemented method of claim 9, further comprising:
generating similarity scores between the overrepresented gene sets for the biological pathways;
generating a graph for the overrepresented gene sets, wherein the graph comprises vertices indicating the overrepresented gene sets for the biological pathways and edges indicating the similarity scores; and
generating, utilizing a community detection model, a community of similar gene sets from the graph for the overrepresented gene sets.

11. The computer-implemented method of claim 10, further comprising:
generating a filtered community of similar genes by filtering, utilizing a minimum redundance maximum relevance feature selection, one or more genes from the community of similar genes; and
generating the gene signature for the target compound from the filtered community of similar genes.

12. A system comprising:
at least one processor; and
at least one non-transitory computer-readable storage medium storing instructions that, when executed by the at least one processor, cause the system to:
generate, utilizing a differential expression model, a first set of gene differential expression measures for a plurality of genes in relation to sensitivity from applying a target compound in a first pre-clinical dataset corresponding to a first data modality;
generate, utilizing the differential expression model, a second set of gene differential expression measures for the plurality of genes in relation to sensitivity from applying the target compound to a second pre-clinical dataset corresponding to a second data modality;
generate a multi-modal ranked set of genes from the first set of gene differential expression measures and the second set of gene differential expression measures; and
generate, from the multi-modal ranked set of genes utilizing an overrepresentation analysis model, a gene signature for the target compound, wherein the gene signature comprises a subset of genes from the multi-modal ranked set of genes that correlate with gene expression sensitivity to the target compound.

13. The system of claim 12, further comprising instructions that, when executed by the at least one processor, cause the system to generate the first set of gene differential expression measures by:
generating area under the curve metrics from dose-response curves in applying the target compound in the first pre-clinical dataset; and
generating the first set of gene differential expression measures from the area under the curve metrics.

14. The system of claim 12, further comprising instructions that, when executed by the at least one processor, cause the system to:
determine area under the curve metrics or inhibitory concentration metrics for samples from the first pre-clinical dataset and the second pre-clinical dataset; and
generate the first set of gene differential expression measures by applying sensitivity weights to a subset of samples from the first pre-clinical dataset and the second pre-clinical dataset based on the area under the curve metrics or the inhibitory concentration metrics.

15. The system of claim 12, further comprising instructions that, when executed by the at least one processor, cause the system to generate the multi-modal ranked set of genes by:
generating a first ranked list of genes and a second ranked list of genes by ranking the first set of gene differential expression measures and the second set of gene differential expression measures; and
combining the first ranked list of genes and the second ranked list of genes to generate the multi-modal ranked set of genes,
wherein combining the first ranked list of genes and the second ranked list of genes to generate the multi-modal ranked set of genes comprises applying a bias weight to the first ranked list of genes relative or to the second ranked list of genes.

16. The system of claim 15, further comprising instructions that, when executed by the at least one processor, cause the system to generate the multi-modal ranked set of genes by:
generating an initial multi-modal ranked set of genes from the first ranked list of genes and the second ranked list of genes;
determining statistical significance measures for the initial multi-modal ranked set of genes by comparing the initial multi-modal ranked set of genes to a null distribution of ranks; and
filtering one or more genes from the initial multi-modal ranked set of genes utilizing the statistical significance measures to generate the multi-modal ranked set of genes.

17. A non-transitory computer-readable medium storing instructions that, when executed by at least one processor, cause a computing device to:
generate, utilizing a differential abundance model, a first set of differential abundance measures for a plurality of biomolecules in relation to sensitivity from applying a target compound in a first pre-clinical dataset corresponding to a first data modality;
generate, utilizing the differential abundance model, a second set of differential abundance measures for the plurality of biomolecules in relation to sensitivity from applying the target compound to a second pre-clinical dataset corresponding to a second data modality;
generate a multi-modal ranked set of biomolecules from the first set of differential abundance measures and the second set of differential abundance measures; and
generate, from the multi-modal ranked set of biomolecules utilizing an overrepresentation analysis model, a biomolecule signature for the target compound, wherein the biomolecule signature comprises a subset of biomolecules from the multi-modal ranked set of biomolecules that correlate with biomolecule abundance sensitivity to the target compound.

18. The non-transitory computer-readable medium of claim 17, further comprising instructions that, when executed by the at least one processor, cause the computing device to:
determine area under the curve metrics or inhibitory concentration metrics for samples from the first pre-clinical dataset and the second pre-clinical dataset; and
generate the first set of differential abundance measures by applying sensitivity weights to a subset of samples from the first pre-clinical dataset and the second pre-clinical dataset based on the area under the curve metrics or the inhibitory concentration metrics.

19. The non-transitory computer-readable medium of claim 17, further comprising instructions that, when executed by the at least one processor, cause the computing device to generate the multi-modal ranked set of biomolecules by:
generating a first ranked list of biomolecules and a second ranked list of biomolecules by ranking the first set of differential abundance measures and the second set of differential abundance measures; and
combining the first ranked list of biomolecules and the second ranked list of biomolecules to generate the multi-modal ranked set of biomolecules,
wherein combining the first ranked list of biomolecules and the second ranked list of biomolecules to generate the multi-modal ranked set of biomolecules comprises applying a bias weight to the first ranked list of biomolecules relative or to the second ranked list of biomolecules.

20. The non-transitory computer-readable medium of claim 19, further comprising instructions that, when executed by the at least one processor, cause the computing device to generate the multi-modal ranked set of biomolecules by:
generating an initial multi-modal ranked set of biomolecules from the first ranked list of biomolecules and the second ranked list of biomolecules;
determining statistical significance measures for the initial multi-modal ranked set of biomolecules by comparing the initial multi-modal ranked set of biomolecules to a null distribution of ranks; and
filtering one or more biomolecules from the initial multi-modal ranked set of biomolecules utilizing the statistical significance measures to generate the multi-modal ranked set of biomolecules.
